(19) United States
(12) Patent Application Publication
(10) Pub. No.: US 2025/0132047 A1
(43) Pub. Date: Apr. 24, 2025

(54) Methods to Simulate Prospective Embryo Genotypes and Approximate Disease Occurrence Risk

(71) Applicant: MYOME, INC., MENLO PARK, CA (US)

(72) Inventors: AKASH KUMAR, PALO ALTO, CA (US); KATE IM, ELLICOTT CITY, MD (US); MATTHEW RABINOWITZ, SAN FRANCISCO, CA (US)

(73) Assignee: MYOME, INC., MENLO PARK, CA (US)

(21) Appl. No.: 18/695,411

(22) PCT Filed: Sep. 27, 2022
(86) PCT No.: PCT/US2022/077123

§ 371 Date: Mar. 26, 2024

Related U.S. Application Data
(60) Provisional application No. 63/248,749, filed on Sep. 27, 2021.

(51) Int. Cl. G16H 50/30; C12Q 1/6869; C12Q 1/6883; G16B 20/20; G16B 30/00; G16H 50/50

(52) U.S. Cl. CPC G16H 50/30; C12Q 1/6869; C12Q 1/6883; G16B 20/20; G16B 30/00; G16H 50/50; C12Q 2600/156

(57) ABSTRACT

Disclosed herein are methods of determining a probability of disease distribution associated with a prospective embryo by generating phased parental chromosomes, determining one or more meiotic recombination sites of interest, and generating one or more simulated embryo genotypes. A polygenic risk model may be applied to each simulated embryo genotype to generate a polygenic risk scores and determine a probability of disease distribution for one or more diseases for the prospective embryo.

### CROSS REFERENCE TO RELATED APPLICATIONS

This application claims the benefit of U.S. Provisional Application No. 63/248,749, filed on Sep. 27, 2021, which is incorporated herein by reference in its entirety.

### TECHNOLOGICAL FIELD

The present disclosure relates in general to determining disease risk, and more specifically, to methods for determining a disease occurrence risk for prospective embryos.

### BACKGROUND

Currently, in vitro fertilization (IVF) clinics test for aneuploidies and single gene disorders that are known to run in families. However, one in every two couples have a family history of common diseases which is impacted by a combination of genetic, environmental, and lifestyle risk factors. Moreover, currently sperm donor clinics test for propensity to develop a subset of diseases caused by single gene disorders but fail to consider the possibility of a future embryo developing complex, polygenic diseases.

### BRIEF SUMMARY

As described herein, example embodiments determine a probability of disease distribution associated with a prospective embryo. A number of simulated embryo genotypes are generated and then used to generate a polygenic risk score set, which in turn enables determination of a probability of disease distribution for one or more diseases for the prospective embryo.

Example embodiments described herein allow the estimation of a disease occurrence or recurrence associated with a future prospective embryo created via IVF. Currently, while certain methods may infer the genotype of an existing embryo based on phased parental genomes and using microarray genotyping of the existing embryo, such methods are not informative with regards to the chance of occurrence or reoccurrence of particular diseases associated with future embryos. Given the financial and physical costs associated with IVF, it may be advantageous to consider the likelihood of disease(s) for a prospective embryo prior to undergoing IVF such that all parties may make a more informed decision about the course of action to pursue. This may be of particular interest for individuals with personal and/or family history of complex disease.

One possible approach to predicting disease risk is to simulate possible embryos using an unlinked approximation method. With an unlinked approximation method, each site in the embryo genotype may be treated independently from other sites. For example, to simulate an embryo genotype, the probability of each genotype from a respective parent may be determined and used to construct a simulated embryo genotype.

The unlinked approximation method is relatively computationally simple and fast and may produce satisfactory results in some instances. However, the unlinked approximation method suffers from several drawbacks in instances where linkage between sites in the embryo genotype is important. In particular, since unlinked approximation methods do not account for parental chromosomes being inherited in large segments, such approximation methods underestimate the genetic variability between sibling embryos, which in turn leads to an underestimation of the variability in disease risk and variability in genetic ancestry between sibling embryos. As embryos inherit half their DNA from either parent, their ancestry as quantified by principal components falls halfway in between the parent's ancestry, on average. However, there is substantial variability around this average, leading to variation in genetic ancestry between siblings, which is missed in the unlinked approximation.

Furthermore, non-additive effects across nearby genetic sites (e.g., epistasis) or across haplotypes (e.g., dominance) play an important role in contribution to disease risk. For example, one special case of this in an oligogenic context is compound heterozygosity, where two recessive alleles can either have no effect or be disease causing, depending on whether the two alleles are inherited from the same parent or from different parents.

Embodiments described herein advantageously allow for the determination of a disease risk for a prospective embryo using a linked approximation method. In particular, parental chromosomes (e.g., paternal chromosomes and maternal chromosomes) may be phased to obtain a paternal genotype and maternal genotype. In some embodiments, genomic information from a sibling embryo (e.g., from a prior round of IVF) may also be determined. In the linked approximation, meiotic recombination sites of interest may be inferred based on the parental chromosomes and genomic information from the sibling embryo. Parental gametes may then be simulated based on the respective phased parental chromosomes and the meiotic recombination sites of interest and subsequently used to generate simulated embryo genotypes. Therefore, linked approximation methods advantageously allow for the simulation of embryo genotypes which inherit chromosomal segments from each parent and allow for chromosome-length parental haplotypes to be determined across an entire genome for a simulated embryo. This allows for preservation of parental ancestry and leads to increased accuracy in the genetic variability of a simulated embryos. Consideration of linkage may be particularly important when considering polygenic risk models which include high-effect linked single nucleotide polymorphisms (SNPs), such as autoimmune conditions. As such, subsequent polygenic risk scoring may be performed on the simulated embryos to yield a more accurate probability of disease distribution for a prospective embryo.

As such, methods are provided herein for determining a probability of disease distribution associated with a prospective embryo. The method comprises generating a phased maternal chromosome set and a phased paternal chromosome set and determining one or more meiotic recombination sites of interest. The method further comprises generating one or more simulated embryo genotypes based on the phased maternal chromosome set, the phased paternal chromosome set, and the one or more meiotic recombination sites of interest. The method further comprises applying a polygenic risk model to the one or more simulated embryo genotypes to generate a polygenic risk score set, wherein the polygenic risk score set includes a polygenic risk score for each simulated embryo genotype of the one or more simulated embryo genotypes and determining a probability of disease distribution for one or more diseases for the prospective embryo based on the polygenic risk score set.

In some embodiments, the method further comprises converting each polygenic risk score to a relative risk of disease based on the polygenic risk score. In some embodiments, converting each polygenic risk score to the relative risk of disease further comprises calculating, using an effect size model, an odds ratio for the polygenic risk score and determining the relative risk of disease based on the odds ratio and a prevalence of disease associated with a particular disease.

In some embodiments, the method further comprises determining one or more risk thresholds for each disease. In some embodiments, the method further comprises determining a percentage of the probability of disease distribution for a disease which satisfies the one or more risk thresholds corresponding to the disease.

In some embodiments, the method further comprises normalizing, based on population data, each polygenic risk score in the polygenic risk score set to produce a normalized polygenic risk score set, wherein determining the probability of disease distribution is based on the normalized polygenic risk score set. In some embodiments, population data comprises ancestry specific population data.

In some embodiments, the method further comprises generating, using a meiotic recombination model, a maternal gamete based on the phased maternal chromosome set and the one or more meiotic recombination sites of interest. In some embodiments, the method further comprises generating, using the meiotic recombination model, a paternal gamete based on the phased paternal chromosome set and the one or more meiotic recombination sites of interest. In some embodiments, the method further comprises generating the one or more simulated embryo genotypes based on the paternal gamete and the maternal gamete.

In some embodiments, the method further comprises obtaining a maternal genome from a maternal subject and a paternal genome from a paternal subject. In some embodiments, the method further comprises phasing the maternal genome to generate the phased maternal chromosome set. In some embodiments, the method further comprises phasing the paternal genome to generate the phased paternal chromosome set. In some embodiments, phasing of the maternal genome or paternal genome is performed using one or more of population-based methods or molecular based methods.

In some embodiments, the method further comprises performing whole genome sequencing on a biological sample obtained from the maternal subject to determine the maternal genome. In some embodiments, the method further comprises performing whole genome sequencing on a biological sample obtained from the paternal subject to determine the paternal genome.

In some embodiments, the method further comprises determining sibling genomic information. In some embodiments, the method further comprises generating the phased maternal chromosome set based on the maternal genome and the sibling genomic information. In some embodiments, the method further comprises generating the phased paternal chromosome set based on the paternal genome and the sibling genomic information.

In some embodiments, chromosome-length parental haplotypes are obtained across an entire genome for each simulated embryo.

In some embodiments, the method further comprises obtaining population genotype data comprising individual genotypes for a plurality of unrelated individuals. In some embodiments, the method further comprises generating the phased maternal chromosome set based on the maternal genome and the population genotype data. In some embodiments, the method further comprises generating the phased paternal chromosome set based on the paternal genome and the population genotype data.

In some embodiments, the method further comprises determining sibling genomic information. In some embodiments, the method further comprises determining the one or more meiotic recombination sites of interest based on the sibling genome, the maternal genome, and the paternal genome.

In some embodiments, sibling genomic information is determined using at least one of array measurements, next-generation sequencing, or whole genome sequencing, and sibling genomic information is obtained from at least one of a sibling embryo, a full biological sibling, or a half biological sibling.

In some embodiments, the method further comprises generating an additional in-vitro fertilization (IVF) cycle recommendation based on the probability of disease distribution for one or more diseases for the prospective embryo. In some embodiments, the method further comprises outputting the IVF cycle recommendation. In some embodiments, the additional IVF cycle recommendation is indicative of whether to perform an additional round of IVF. In some embodiments, the method further includes determining a disease occurrence risk based on the probability of disease distribution, wherein the IVF cycle recommendation is based on the disease occurrence risk.

Similarly, an apparatus for determining a probability of disease distribution associated with a prospective embryo is disclosed herein. The example apparatus comprises a processor and a memory storing software instructions that, when executed by the processor, cause the apparatus to generate a phased maternal chromosome set and a phased paternal chromosome set and determine one or more meiotic recombination sites of interest The processor and memory storing software instructions that, when executed by the processor, further cause the apparatus to generate one or more simulated embryo genotypes based on the phased maternal chromosome set, the phased paternal chromosome set, and the one or more meiotic recombination sites of interest. The processor and memory storing software instructions that, when executed by the processor, further cause the apparatus to apply a polygenic risk model to the one or more simulated embryo genotypes to generate a polygenic risk score set, wherein the polygenic risk score set includes a polygenic risk score for each simulated embryo genotype of the one or more simulated embryo genotypes and determine a probability of disease distribution for one or more diseases for the prospective embryo based on the polygenic risk score set.

In some embodiments, the processor and the memory storing software instructions that, when executed by the processor, further cause the apparatus to convert each polygenic risk score to a relative risk of disease based on the polygenic risk score. In some embodiments, the processor and the memory storing software instructions that, when executed by the processor when converting each polygenic risk score to the relative risk of disease, further cause the apparatus to calculate, using an effect size model, an odds ratio for the polygenic risk score and determine the relative risk of disease based on the odds ratio and a prevalence of disease associated with a particular disease.

In some embodiments, the processor and the memory storing software instructions that, when executed by the processor, further cause the apparatus to determine one or more risk thresholds for each disease. In some embodiments, the processor and the memory storing software instructions that, when executed by the processor, further cause the apparatus to determine a percentage of the probability of disease distribution for a disease which satisfies the one or more risk thresholds corresponding to the disease.

In some embodiments, the processor and the memory storing software instructions that, when executed by the processor, further cause the apparatus to normalize, based on population data, each polygenic risk score in the polygenic risk score set to produce a normalized polygenic risk score set, wherein determining the probability of disease distribution is based on the normalized polygenic risk score set. In some embodiments, population data comprises ancestry specific population data.

In some embodiments, the processor and the memory storing software instructions that, when executed by the processor, further cause the apparatus to generate, using a meiotic recombination model, a maternal gamete based on the phased maternal chromosome set and the one or more meiotic recombination sites of interest. In some embodiments, the processor and the memory storing software instructions that, when executed by the processor, further cause the apparatus to generate, using the meiotic recombination model, a paternal gamete based on the phased paternal chromosome set and the one or more meiotic recombination sites of interest. In some embodiments, the processor and the memory storing software instructions that, when executed by the processor, further cause the apparatus to generate the one or more simulated embryo genotypes based on the paternal gamete and the maternal gamete.

In some embodiments, the processor and the memory storing software instructions that, when executed by the processor, further cause the apparatus to obtain a maternal genome from a maternal subject and a paternal genome from a paternal subject. In some embodiments, the processor and the memory storing software instructions that, when executed by the processor, further cause the apparatus to phase the maternal genome to generate the phased maternal chromosome set. In some embodiments, the processor and the memory storing software instructions that, when executed by the processor, further cause the apparatus to phase the paternal genome to generate the phased paternal chromosome set. In some embodiments, phasing of the maternal genome or paternal genome is performed using one or more of population-based methods or molecular based methods.

In some embodiments, the processor and the memory storing software instructions that, when executed by the processor, further cause the apparatus to perform whole genome sequencing on a biological sample obtained from the maternal subject to determine the maternal genome. In some embodiments, the processor and the memory storing software instructions that, when executed by the processor, further cause the apparatus to perform whole genome sequencing on a biological sample obtained from the paternal subject to determine the paternal genome.

In some embodiments, the processor and the memory storing software instructions that, when executed by the processor, further cause the apparatus to determine sibling genomic information. In some embodiments, the processor and the memory storing software instructions that, when executed by the processor, further cause the apparatus to generate the phased maternal chromosome set based on the maternal genome and the sibling genomic information. In some embodiments, the processor and the memory storing software instructions that, when executed by the processor, further cause the apparatus to generate the phased paternal chromosome set based on the paternal genome and the sibling genomic information.

In some embodiments, chromosome-length parental haplotypes are obtained across an entire genome for each simulated embryo.

In some embodiments, the processor and the memory storing software instructions that, when executed by the processor, further cause the apparatus to obtain population genotype data comprising individual genotypes for a plurality of unrelated individuals. In some embodiments, the processor and the memory storing software instructions that, when executed by the processor, further cause the apparatus to generate the phased maternal chromosome set based on the maternal genome and the population genotype data. In some embodiments, the processor and the memory storing software instructions that, when executed by the processor, further cause the apparatus to generate the phased paternal chromosome set based on the paternal genome and the population genotype data.

In some embodiments, the processor and the memory storing software instructions that, when executed by the processor, further cause the apparatus to determine sibling genomic information. In some embodiments, the processor and the memory storing software instructions that, when executed by the processor, further cause the apparatus to determine the one or more meiotic recombination sites of interest based on the sibling genome, the maternal genome, and the paternal genome.

In some embodiments, sibling genomic information is determined using at least one of array measurements, next-generation sequencing, or whole genome sequencing, and sibling genomic information is obtained from at least one of a sibling embryo, a full biological sibling, or a half biological sibling.

In some embodiments, the processor and the memory storing software instructions that, when executed by the processor, further cause the apparatus to generate an additional in-vitro fertilization (IVF) cycle recommendation based on the probability of disease distribution for one or more diseases for the prospective embryo. In some embodiments, the processor and the memory storing software instructions that, when executed by the processor, further cause the apparatus to output the IVF cycle recommendation. In some embodiments, the additional IVF cycle recommendation is indicative of whether to perform an additional round of IVF. In some embodiments, the processor and the memory storing software instructions that, when executed by the processor, further cause the apparatus to determine a disease occurrence risk based on the probability of disease distribution, wherein the IVF cycle recommendation is based on the disease occurrence risk.

In addition, a computer program product for determining a probability of disease distribution associated with a prospective embryo is disclosed herein. The computer program product includes at least one non-transitory computer-readable storage medium storing software instructions that, when executed by an apparatus, cause the apparatus to generate a phased maternal chromosome set and a phased paternal chromosome set and determine one or more meiotic recombination sites of interest. The computer program product includes at least one non-transitory computer-readable storage medium storing software instructions that, when executed by an apparatus, further cause the apparatus to generate one or more simulated embryo genotypes based on the phased maternal chromosome set, the phased paternal chromosome set, and the one or more meiotic recombination sites of interest. The computer program product includes at least one non-transitory computer-readable storage medium storing software instructions that, when executed by an apparatus, further cause the apparatus to apply a polygenic risk model to the one or more simulated embryo genotypes to generate a polygenic risk score set, wherein the polygenic risk score set includes a polygenic risk score for each simulated embryo genotype of the one or more simulated embryo genotypes and determine a probability of disease distribution for one or more diseases for the prospective embryo based on the polygenic risk score set.

In some embodiments, the computer program product includes at least one non-transitory computer-readable storage medium storing software instructions that, when executed by an apparatus, further cause the apparatus to convert each polygenic risk score to a relative risk of disease based on the polygenic risk score. In some embodiments, the computer program product includes at least one non-transitory computer-readable storage medium storing software instructions that, when executed by an apparatus and when converting each polygenic risk score to the relative risk of disease, further cause the apparatus to calculate, using an effect size model, an odds ratio for the polygenic risk score and determine the relative risk of disease based on the odds ratio and a prevalence of disease associated with a particular disease.

In some embodiments, the computer program product includes at least one non-transitory computer-readable storage medium storing software instructions that, when executed by an apparatus, further cause the apparatus to determine one or more risk thresholds for each disease. In some embodiments, the computer program product includes at least one non-transitory computer-readable storage medium storing software instructions that, when executed by an apparatus, further cause the apparatus to determine a percentage of the probability of disease distribution for a disease which satisfies the one or more risk thresholds corresponding to the disease.

In some embodiments, the computer program product includes at least one non-transitory computer-readable storage medium storing software instructions that, when executed by an apparatus, further cause the apparatus to normalize, based on population data, each polygenic risk score in the polygenic risk score set to produce a normalized polygenic risk score set, wherein determining the probability of disease distribution is based on the normalized polygenic risk score set. In some embodiments, population data comprises ancestry specific population data.

In some embodiments, the the computer program product includes at least one non-transitory computer-readable storage medium storing software instructions that, when executed by an apparatus, further cause the apparatus to generate, using a meiotic recombination model, a maternal gamete based on the phased maternal chromosome set and the one or more meiotic recombination sites of interest. In some embodiments the computer program product includes at least one non-transitory computer-readable storage medium storing software instructions that, when executed by an apparatus, further cause the apparatus to generate, using the meiotic recombination model, a paternal gamete based on the phased paternal chromosome set and the one or more meiotic recombination sites of interest. In some embodiments, the computer program product includes at least one non-transitory computer-readable storage medium storing software instructions that, when executed by an apparatus, further cause the apparatus to generate the one or more simulated embryo genotypes based on the paternal gamete and the maternal gamete.

In some embodiments, the computer program product includes at least one non-transitory computer-readable storage medium storing software instructions that, when executed by an apparatus, further cause the apparatus to obtain a maternal genome from a maternal subject and a paternal genome from a paternal subject. In some embodiments, the computer program product includes at least one non-transitory computer-readable storage medium storing software instructions that, when executed by an apparatus, further cause the apparatus to phase the maternal genome to generate the phased maternal chromosome set. In some embodiments, the computer program product includes at least one non-transitory computer-readable storage medium storing software instructions that, when executed by an apparatus, further cause the apparatus to phase the paternal genome to generate the phased paternal chromosome set. In some embodiments, phasing of the maternal genome or paternal genome is performed using one or more of population-based methods or molecular based methods.

In some embodiments, the computer program product includes at least one non-transitory computer-readable storage medium storing software instructions that, when executed by an apparatus, further cause the apparatus to perform whole genome sequencing on a biological sample obtained from the maternal subject to determine the maternal genome. In some embodiments, the computer program product includes at least one non-transitory computer-readable storage medium storing software instructions that, when executed by an apparatus, further cause the apparatus to perform whole genome sequencing on a biological sample obtained from the paternal subject to determine the paternal genome.

In some embodiments, the computer program product includes at least one non-transitory computer-readable storage medium storing software instructions that, when executed by an apparatus, further cause the apparatus to determine sibling genomic information. In some embodiments, the computer program product includes at least one non-transitory computer-readable storage medium storing software instructions that, when executed by an apparatus, further cause the apparatus to generate the phased maternal chromosome set based on the maternal genome and the sibling genomic information. In some embodiments, the computer program product includes at least one non-transitory computer-readable storage medium storing software instructions that, when executed by an apparatus, further cause the apparatus to generate the phased paternal chromosome set based on the paternal genome and the sibling genomic information.

In some embodiments, chromosome-length parental haplotypes are obtained across an entire genome for each simulated embryo.

In some embodiments, the computer program product includes at least one non-transitory computer-readable storage medium storing software instructions that, when executed by an apparatus, further cause the apparatus to obtain population genotype data comprising individual genotypes for a plurality of unrelated individuals. In some embodiments, the computer program product includes at least one non-transitory computer-readable storage medium storing software instructions that, when executed by an apparatus, further cause the apparatus to generate the phased maternal chromosome set based on the maternal genome and the population genotype data. the computer program product includes at least one non-transitory computer-readable storage medium storing software instructions that, when executed by an apparatus, further cause the apparatus to generate the phased paternal chromosome set based on the paternal genome and the population genotype data.

In some embodiments, the computer program product includes at least one non-transitory computer-readable storage medium storing software instructions that, when executed by an apparatus, further cause the apparatus to determine sibling genomic information. In some embodiments, the computer program product includes at least one non-transitory computer-readable storage medium storing software instructions that, when executed by an apparatus, further cause the apparatus to determine the one or more meiotic recombination sites of interest based on the sibling genome, the maternal genome, and the paternal genome.

In some embodiments, sibling genomic information is determined using at least one of array measurements, next-generation sequencing, or whole genome sequencing, and sibling genomic information is obtained from at least one of a sibling embryo, a full biological sibling, or a half biological sibling.

In some embodiments, the computer program product includes at least one non-transitory computer-readable storage medium storing software instructions that, when executed by an apparatus, further cause the apparatus to generate an additional in-vitro fertilization (IVF) cycle recommendation based on the probability of disease distribution for one or more diseases for the prospective embryo. The computer program product includes at least one non-transitory computer-readable storage medium storing software instructions that, when executed by an apparatus, further cause the apparatus to output the IVF cycle recommendation. In some embodiments, the additional IVF cycle recommendation is indicative of whether to perform an additional round of IVF. The computer program product includes at least one non-transitory computer-readable storage medium storing software instructions that, when executed by an apparatus, further cause the apparatus to determine a disease occurrence risk based on the probability of disease distribution, wherein the IVF cycle recommendation is based on the disease occurrence risk.

The foregoing brief summary is provided merely for purposes of summarizing some example embodiments described herein. Because the above-described embodiments are merely examples, they should not be construed to narrow the scope of this disclosure in any way. It will be appreciated that the scope of the present disclosure encompasses many potential embodiments in addition to those summarized above, some of which will be described in further detail below

### BRIEF DESCRIPTION OF DRAWINGS

Having described certain example embodiments in general terms above, reference will now be made to the accompanying drawings, which are not necessarily drawn to scale. Some embodiments may include fewer or more components than those shown in the figures.

FIG. 1 illustrates an example process overview for generating a polygenic risk score for a simulated embryo genotype, which may be used in accordance with some example embodiments described herein.

FIGS. 2A-2B illustrates example processes for phasing parental genomes using a parental support model, which may be used in accordance with some example embodiments described herein.

FIG. 3 depicts an example hidden Markov model setup, in accordance with some example embodiments described herein.

FIG. 4 depicts an example hidden Markov model calculation, in accordance with some example embodiments described herein.

FIG. 5 depicts an example parental support model framework, in accordance with some example embodiments described herein.

FIG. 6 depicts an operational example of a probability of disease distribution, in accordance with some example embodiments described herein.

FIGS. 7A-7L depict operational examples of probability of disease distribution for a variety of diseases as determined using an unlinked approximation and linked approximation, in accordance with some example embodiments described herein.

FIG. 8 depicts an example of a polygenic risk score distribution for an unlinked approximation and linked approximation, in accordance with some example embodiments described herein.

FIG. 9 depicts an example of ancestry information included in a simulated embryo genotype using an unlinked approximation and linked approximation, in accordance with some example embodiments described herein.

FIG. 10 illustrates a schematic block diagram of example device that may perform various operations in accordance with some example embodiments described herein.

FIG. 11 illustrates an example process for phasing a parental genome, in accordance with some example embodiments described herein.

FIG. 12 illustrates an example process for generating a simulated embryo genotype, in accordance with some example embodiments described herein.

FIGS. 13A-13D depict an example probability of disease distribution for a variety of diseases corresponding to example 7.

FIGS. 14A-14B illustrate disease odds ration by polygenic risk score decile corresponding to example 6.

FIG. 15 illustrates the correlation of polygenic risk score from embryo predictions and a born child.

FIG. 16 illustrates an example plot of transmitted haplotypes for a sibling embryo.

FIG. 17 illustrates an example flowchart for performing one or more actions based on an output of using the linked approximation.

### DETAILED DESCRIPTION

Some example embodiments will now be described more fully hereinafter with reference to the accompanying figures, in which some, but not necessarily all, embodiments are shown. Because inventions described herein may be embodied in many different forms, the invention should not be limited solely to the embodiments set forth herein; rather, these embodiments are provided so that this disclosure will satisfy applicable legal requirements.

Many modifications and other embodiments of the disclosure set forth herein will come to mind to one skilled in the art to which this disclosure pertains having the benefit of the teachings presented in the foregoing description and the associated drawings. Therefore, it is to be understood that the embodiments are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly describe herein are also contemplated as may be set forth in some of the appended claims. Although specific terms are employed herein, they are used in a generic and descriptive sense only and not for purposes of limitation.

### Definition of Certain Terms

Technical and scientific terms used herein have the meanings commonly understood by one of ordinary skill in the art to which the present invention pertains, unless otherwise defined. Materials to which reference is made in the following description and examples are obtainable from commercial sources, unless otherwise noted.

The terms “computer-readable medium” and “memory” refer to non-transitory storage hardware, non-transitory storage device or non-transitory computer system memory that may store computer-executable instructions or software programs that may be accessed by a controller, a microcontroller, a computational system or a module of a computational system. A non-transitory computer-readable medium may be accessed by a computational system or a module of a computational system to retrieve and/or execute the computer-executable instructions or software programs stored on the medium. Exemplary non-transitory computer-readable media may include, but are not limited to, one or more types of hardware memory, non-transitory tangible media (for example, one or more magnetic storage disks, one or more optical disks, one or more USB flash drives), computer system memory or random access memory (such as, DRAM, SRAM, EDO RAM), and the like.

The term “computing device” may refer to any computer embodied in hardware, software, firmware, and/or any combination thereof. Non-limiting examples of computing devices include a personal computer, a server, a laptop, a mobile device, a smartphone, a fixed terminal, a personal digital assistant (“PDA”), a kiosk, a custom-hardware device, a wearable device, a smart home device, an Internet-of-Things (“IoT”) enabled device, and a network-linked computing device.

The term “about” may mean that the number comprehended is not limited to the exact number set forth herein, and is intended to refer to numbers substantially around the recited number while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The term “gene” relates to stretches of DNA or RNA that encode a polypeptide or that play a functional role in an organism. A gene can be a wild-type gene, or a variant or mutation of the wild-type gene. A “gene of interest” refers to a gene, or a variant of a gene, that may or may not be known to be associated with a particular phenotype, or a risk of a particular phenotype.

The term “expression” refers to the process by which a polynucleotide is transcribed from a DNA template (such as into a mRNA or other RNA transcript) and/or the process by which a transcribed mRNA is subsequently translated into peptides, polypeptides, or proteins. Expression of a gene encompasses not only cellular gene expression, but also the transcription and translation of nucleic acid(s) in cloning systems and in any other context. Where a nucleic acid sequence encodes a peptide, polypeptide, or protein, gene expression relates to the production of 20 the nucleic acid (e.g., DNA or RNA, such as mRNA) and/or the peptide, polypeptide, or protein. Thus, “expression levels” can refer to an amount of a nucleic acid (e.g. mRNA) or protein in a sample.

The term “haplotype” refers to a group of genes or alleles that are inherited together, or expected to be inherited together, from a single antecedent (such as a father, mother, grandfather, 25 grandmother, etc.). The term “antecedent” refers to a person from who a subject has descended, or in the case of an embryo from who a potential subject will have descended. In preferred aspects, the antecedent refers to a mammalian subject, such as a human subject.

### Data Collection

Genetic material for analysis by the methods described herein may be obtained from various sources, including somatic cells (e.g., white blood cells, cells from tissue biopsies), germ cells (e.g., sperm, eggs, polar bodies). Genetic material may be collected from genetic relatives of a prospective embryo (e.g., a biological mother, biological father, biological siblings, sibling embryos, grandparents, etc.). In some embodiments, genomic DNA may be extracted from whole blood or saliva samples provided by a paternal subject, maternal subject, sibling subject (e.g., born children), grandparent subject, etc.

### Simulated Embryo Genotype Generation-Linked Approach

As described above, it may be advantageous to use a linked approach to generate simulated embryo genotypes such that chromosome-length parental haplotypes may be determined across an entire genome for a simulated embryo. Additionally, using the linked approach more accurately simulates the range of possible genotypes (and thus PRS scores) amongst sibling embryos and maintains genomic ancestry composition (which is lost using unlinked genotypes), thereby allowing for local ancestry approaches to be applied to risk scoring. In some embodiments, certain operations of the linked approximation may be performed according to the methods in “Whole-genome risk prediction of common diseases in human preimplantation embryos.” Nat Med 28, 513-516 (2022). to Kumar et al., published on Mar. 21, 2022, which is herein incorporated by reference in its entirety.

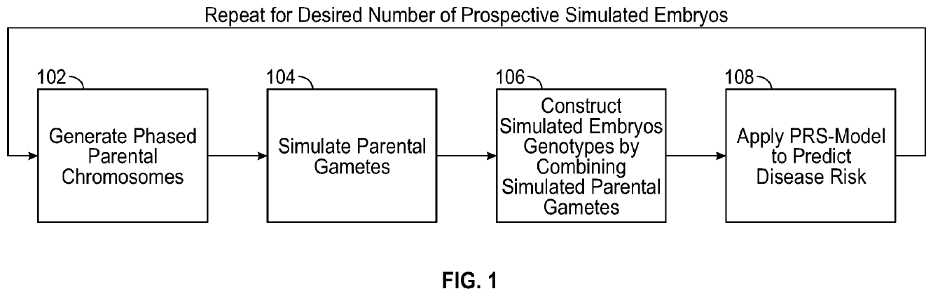

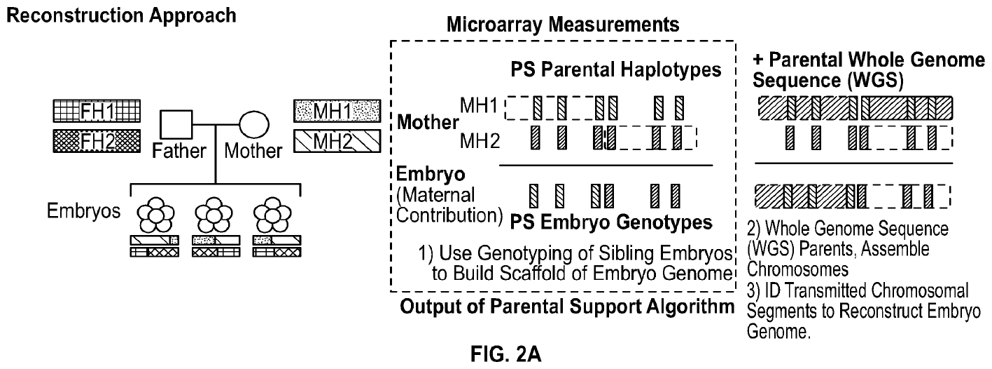

FIG. 1 outlines the various operations performed for generating simulated embryo genotypes and subsequently predicting a probability of disease distribution for a prospective embryo. These operations are outlined in further detail below. Operations 102-106 may be performed to yield a simulated embryo genotype representative of a possible genotype for a prospective embryo. Operation 108 may then be performed on the simulated embryo genotype to determine a PRS score (e.g., disease risk) for the simulated embryo genotype. Operations 102-108 may be repeated a desired number of times such that one or more simulated embryo genotypes may be generated for a prospective embryo. In some embodiments, a threshold number of simulated embryo genotypes may be required. In some embodiments, at least ten or more simulated embryo genotypes may be required. PRS may then be generated for each simulated embryo genotype and the PRSs may be used to determine a probability of disease distribution for the prospective embryo.

### Sequencing

Various molecular based methods of phasing are well known in the art and may be used to implement the methods described herein unless dictated otherwise by context. Shotgun sequencing refers to a method of sequencing random DNA strands from a genome or large genetic sample. DNA is broken up randomly into numerous small segments, which are sequenced (e.g., using the chain termination method) to obtain reads. Multiple overlapping reads for the target DNA are obtained by performing several rounds of this fragmentation and sequencing. Computational algorithms then use the overlapping ends of different reads to assemble the reads of the random segments into a continuous sequence. Shotgun sequencing may be used for whole genome sequencing. Any suitable form of sequencing, including those describe herein, may be used to identify variants (e.g., SNPs) in a subject which may subsequently be used as the basis for measuring genetic signals indicative of ploidy status for a chromosomal segment comprising that variant, as described elsewhere herein. According to certain aspects of the invention, hierarchical sequencing may be used for whole genome sequencing. In some embodiments, phasing of parental genome sequences may be performed according to the methods in WO 2021/067417 to Kumar et al., published on Apr. 8, 2021, which is herein incorporated by reference in its entirety.

In some embodiments, DNA sequencing may comprise for example Sanger sequencing (chain-termination sequencing). DNA sequencing may comprise use of next-generation sequencing (NGS) or second generation sequencing technology, which is typically characterized by being highly scalable, allowing an entire genome to be sequenced at once. NGS technology generally allows multiple fragments to be sequenced at once allowing for “massively parallel” sequencing in an automated process. DNA sequencing may comprise third generation sequencing technology (e.g., nanopore sequencing or SMRT sequencing), which generally allows for obtaining longer reads than obtainable via second generation sequencing technology. Sequencing may comprise paired-end sequencing, where feasible, in which both ends of a DNA fragment are sequenced, which may improve the ability to align the reads to a longer sequencing. DNA sequencing may comprise sequencing by synthesis/ligation (e.g., ILLUMINA® sequencing), single-molecule real time (SMRT) sequencing (e.g., PACBIO® sequencing), nanopore sequencing (e.g., OXFORD NANOPORE® sequencing), ion semiconductor sequencing (Ion Torrent sequencing), combinatorial probe anchor synthesis sequencing, pyrosequencing, etc.

In some aspects, phasing uses data generated from linked-read sequencing, long fragment reads, fosmid-pool-based phasing, contiguity preserving transposon sequencing, whole genome sequencing, Hi-C methodologies, dilution-based sequencing, targeted sequencing (including HLA typing), or microarray.

Some aspects include the use of sparse phased genotypes obtained independently to provide a scaffold to guide phasing. Computer software such as HapCUT, SHAPEIT, MaCH, BEAGLE or EAGLE can be used to phase an antecedent's genotype.

Population based phasing may use a reference panel such as 1000 Genomes or Haplotype Reference Consortium to phase the genotype. In some instances, phasing accuracy may be improved by the addition of genotype data from relatives such as grandparents, siblings, or children.

### Phased Parental Genotype Generation

To begin the process for generating a simulated embryo genotype, a phased maternal chromosome set and phased paternal chromosome set may be generated for the maternal and paternal subject, respectively. A respective chromosome set may include one or more chromosomes corresponding to a homologous chromosome pair. The phased maternal chromosome set and phased paternal chromosome set may each be generated by phasing the genome associated with the maternal subject and paternal subject, respectively, using various methods such as populated based and/or molecular based methods as described above.

Both the maternal genome and paternal genome may be fully phased. A respective parental genome may be phased using whole genome sequencing (WGS). In some embodiments, each parental genome is phased using a parental support model. The parental support model may describe a method of combining SNP array measurements from one or more existing embryos and the parents along with recombination frequencies from a database (e.g., HapMap) to enable accurate prediction of chromosome copy numbers, insertions and deletions, embryo genotypes, parent haplotypes as well as embryo parent haplotype origin hypotheses using method similar to those described in U.S. Pat. No. 8,515,679 to Rabinowitz et al., which is herein incorporated by reference in its entirety. The parental support model may include one or more meiotic recombination models, which simulate meiotic recombination sites during meiosis for a respective parental gamete.

For whole parental genome reconstruction, two sources of data are required. Firstly, whole genome sequencing of prospective parents is needed as described below. Secondly, sibling genomic information is also needed. Sibling genomic information may be obtained in a variety of ways. In some embodiments, sibling genomic information may be obtained from sibling embryos by SNP microarray genotype, next-generation sequencing (NGS), etc. In some embodiments, sibling genomic information may be obtained from full biological siblings or half biological siblings, such as by WGS. Although the sibling genomic information may be described herein as being determined with respect to sibling embryos in some exemplary embodiments, it will be appreciated by one of skill in the art that alternative sources for sibling genomic information such as full biological siblings and/or half biological siblings may be used additionally or alternatively to sibling embryos. When SNP microarray genotyping is used to determine sibling genomic information, amplification is required since embryo biopsies yield a limited amount of DNA.

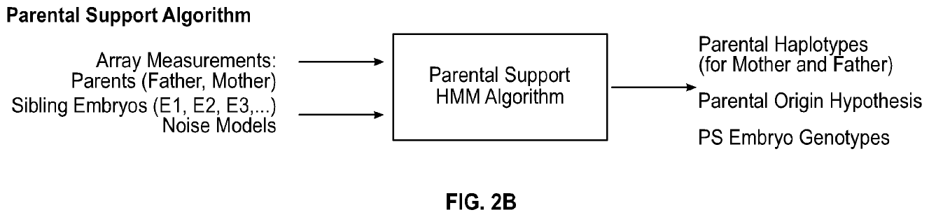

Sibling genomic data is depicted in FIG. 2A. In FIG. 2A, allele measurements at each SNP are pattern-coded based on the parental haplotype of origin in this example.

As illustrated in FIG. 2B, the parental support model may receive and process the data sources (e.g., the WGS from the parents and the SNP microarray genotyping (e.g., genomic information) from the one or more sibling embryos) to generate one or more outputs. The one or more outputs may include the phased parental genome (e.g., both the phased maternal genome and phased paternal genome), a parental origin hypothesis, and the sibling embryo genotypes. The parental support model may be a hidden Markov model (HMM) which accounts for measurements on sibling genotypes as well as parental genotypes to improve accuracy across several hundred thousand positions. Table 1 further outlines the parental support model inputs and outputs.

TABLE 1

| Parental | Parental | Mother (MD) and father (FD) microarray data, with genotypes |
|---|---|---|
| Support | Microarray | marked as AA, AB, BB (A = Ref, B = Mut). |
| Microarray | Data | |
| Inputs | Embryo | Up to k Embryo microarray measurements (ED₁, . . . , EDₖ), stored as |
| | Microarray | intensity values on the “A” channel and “B” channel. |
| | Data | |
| | Denote the full set of data below as: D = (D₁, . . . , Dₙ) = (MDₜ, FDₜ, EDₜⱼ), | |
| | t = 1, . . . , n, j = 1, . . . , k. | |
| Parental | PS Parental | A best estimate of each phased parental genotype, denoted as |
| Support | Haplotypes | (genotype at haplotype1, genotype at haplotype 2). |
| Outputs | | E.g. phased genotype AB signifies that A comes from the first |
| | | haplotype and B from the second. For each position on the |
| | | microarray, the possible phased parental haplotypes are {AA, AB, |
| | | BA, BB}. |
| | Embryo | The particular haplotypes inherited from the specific parent. |
| | Parental | Denoted as H1 or H2, depending on which haplotype the embryo |
| | Origin | inherited from a parent. |
| | Hypothesis | E.g. MH1, FH2 means that the embryo has inherited its mother's |
| | | haplotype 1 and father's haplotype 2. |
| | PS Embryo | A direct combination of mother, father haplotypes and parental |
| | Genotype | origin hypothesis. |
| | | E.g. if parent haplotypes are (BA, AB) and parent origin hypotheses |
| | | are (H1, H2), the embryo inherits a B from mother (H1 from BA) |
| | | and a B from father (H2 from AB), so the PS embryo genotype is |
| | | BB. |

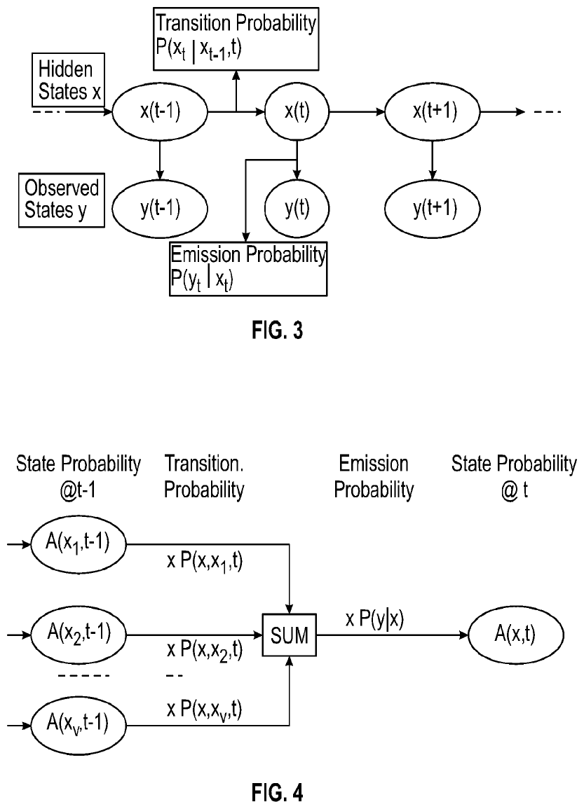

FIG. 3 illustrates an example parental support model setup and FIG. 4 illustrates the parental support model output. In some embodiments, the full implementation of the parental support model supporting meiotic crossovers involves a HMM with a forward-backward (FBA) algorithm implemented.

A HMM is a statistical Markov model in which the system being modeled is assumed to be a Markov process Xₜ, through “time” t, with unobservable (i.e., hidden) states {x}. The approach assumes that there is another process Yₜ, with observable states {y}, whose behavior through time depends on X. The goal is to learn about X by observing Y. In an HMM it may be assumed that for each time instance t, the conditional distribution of Yₜ depends only on X, via probability P(y|x)=P(Yₜ=y|Xₜ=x). This probability is the emission probability. The probability of the observable sequence Y=(Y₁, . . . , Yₙ) can be written by Bayes rule as P(Y)=Σ_{X}P(Y|X)P(X).

FIG. 4 further depicts the posterior probability P(Xₜ=x|(y₁, . . . , yₙ)), i.e. a probability of an unobservable state x at time t, given observed states (y₁, . . . , yₙ). The forward algorithm calculates the joint probability of a hidden state x and (y₁, . . . , yₜ) A(x,t)=P(Xₜ=x, y₁, . . . , yₜ) as A(x,t)=P(Yₜ=yₜ|Xₜ=x)*Σ_{z}P(x|z,t)*A(z,t−1) thus reducing the problem of order/to the problem of order t−1, as seen in FIG. 3. P(x|z,t) is referred to as a hidden state transition probability at time t. Posterior probability of any hidden state x at time n is then P(x|(y₁, . . . , yₙ))˜A(x,n).

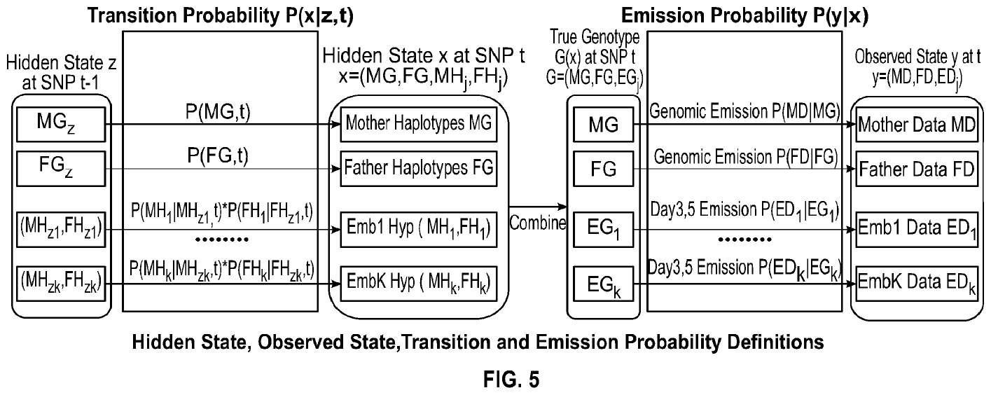

Given the above, FIG. 5 depicts the HHM framework for the parental support model. For the parental support model, the fact that an embryo will inherit alleles from the same parent homolog on consecutive SNPs in incorporated, unless a meiotic recombination (with probability estimated from a database, such as HapMap) has occurred between the two SNPs. The joint distribution of genotype probabilities thus combines the array data, the individual embryo genotypes suggested by the array data, and the parent haplotyping that could produce those distributions of genotypes among various embryos. Consecutive SNPs represent “time” 1. The approach is applied to each full chromosome separately, at all sites on the array. The number of SNPs per chromosome may ranges from around 4,300 (e.g., chromosome 21) to 23,700 (e.g., chromosome 2). The approach may be run across the entire chromosome instead of smaller regions of the genome. This allows for crossovers within, as well as between bins, as well as inference of problematic genome sections.

Table 2 below further illustrates the various parameters and outputs from the parental support model illustrated in FIG. 5.

TABLE 2

| | |
|---|---|
| Observed State | yₜ is the combination of parent and embryo microarray measurements at a |
| | particular SNP, yₜ = (MDₜ, FDₜ, EDₜⱼ) for j = 1, . . . , k. |
| Hidden State | x is defined as a combination of true parent haplotypes and parent origin |
| | hypotheses x = (MG, FG, MH₁, FH₁, . . . , MH_{K}, FHₖ) where |
| | MG, FG∈{AA, AB, BA, BB}, the set of parent haplotype pairs and |
| | MHⱼ, FHⱼ∈{H1, H2}, the set of parental origin hypotheses. |
| Transition | The product of haplotype prior frequencies (assuming no parent SNP linkage) |
| probability | and origin hypotheses transition probabilities. Specifically, for states |
| | x = (MG, FG, MHⱼ, FHⱼ, j = 1 . . . k) at SNP t and state z = (MG_{z}, FG_{z}, MH_{zj}, FH_{zj}, j = 1 . . . k) |
| | at time t − 1 |
| Emission | P(y\|x) is defined as a product of parent and embryo SNP data likelihood |
| probability | (emission probabilities), given the true state x |
| | P(y\|x) = P(MD\|MG) × P(FD\|FG) × Π_{j=1, . . . , k}P(EDⱼ\|EGⱼ), |
| | where EGⱼ is the jth embryo genotype, a direct combination of parent haplotypes |
| | MG, FG and jth parent origin hypotheses (MHⱼ, FHⱼ), as mentioned before. |
| | P(MD\|MG), P(FD\|FG) are parent data genomic emission models and P(ED\|EG) |
| | is the embryo data emission model, further discussed below. |
| Output: PS | Parent haplotypes probability is the marginal of the joint probability P(x\|y), for |
| Parental | all states having specific parent haplotype, i.e. P(MG\|y) = Σ_{x∈MG}P(x\|y). For each |
| Haplotype | parent, we derive the best answer g* as the one maximizing this probability, i.e. |
| | g* = argmax_{g} P(g\|y), with confidence P(g*). |
| Output: | The state maximizing the marginal parent origin hypothesis probability |
| Parental | P(H\|y) = Σ_{x∈H}P(x\|y). |
| Origin Hyp. | |

The transition probability depicted in FIG. 4 may be used to model the meiotic recombinations between consecutive SNPs. The transition probability from state z at SNP t−1 to state x at SNP t is modeled as:

$$P ( x at {SNP}_{t} \vert z at {SNP}_{t - 1} ) = P ( MG , t ) * P ( FG , t ) * {\prod }_{j = 1 , … k} P ( {MH}_{j} \vert {MH}_{zj} , t ) * P ( {FH}_{j} \vert {FH}_{zj} , t ) \qquad ( 1 )$$

In equation 1, P(MG,t) and P(FG,t) are parent haplotype population priors at SNP t derived from a large set of training data and allele frequency public databases. P(MH|MH_{z},t) and P(FH|FH_{z},t) are the hypotheses transition probabilities, and are derived via crossover probabilities between SNPs t−1 and t from a database (e.g, HapMap) simulating a chance of meiotic crossover between SNPs. Specifically, the transition probabilities may be expressed as P(H1|H1,t)=P(H2|H2,t)=1−cₜ (e.g., no crossover occurred) and P(H1|H2,t)=P(H2|H1,t)=cₜ (e.g., crossover occurred), where cₜ is the crossover probability between SNPs t−1 and t.

The emission probability also depicted in FIG. 4 may be used to account for noise in microarray measurements in sequencing parent or sibling samples. Specifically, the emission probabilities are the per SNP product of per channel data likelihood given a true genotype G: P(Data|genotype=G)=P(Data on channel A|G)*P(Data on channel B|G). Two different approaches may be used to model channel data likelihood. In the first approach, a simplified discrete emission model is used.

For the discrete emission model, a channel independent matrix product is obtained using equation 2:

$${F}_{din , dout} ( g , G ) = P ( genotype = g \vert true genotype = G , din , dout ) = P ( \# A ( g ) \vert \# A ( G ) ) * P ( \# B ( g ) \vert \# B ( G ) ) \qquad ( 2 )$$

Here, din is drop in rate and dout is a drop out rate. The product is based on number of alleles A,B in a true genotype G and measured genotype g, as shown in Table 3. Dropin (din) and dropout (dout) rate parameters are fit on a case-by-case basis using microarray intensity data. In some embodiments, the genomic data dropin rate may be set to 0.1% and the genomic data dropout rate may be set to 0.15%.

TABLE 3

| | |
|---|---|
| # alleles in G | #alleles in g (measured) |

| (true) | 0 | 1 | 2 |
|---|---|---|---|
| 0 | 1-din | din | din |
| 1 | dout | 1-dout | 1-dout |
| 2 | dout | dout | 1-dout |

The second approach is a more complex continuous emission model. For the continuous emission model, a two-dimensional likelihood P(Data|G)=P(Channel A Measurement|G)*P(Channel B Measurement|G) is used, where each channel likelihood is parameterized via known, continuous distribution for a given genotype G. Distribution parameters are fitted in each couple using embryo microarray measurements for parental context resulting in genotype G.

The resulting output from the parental support model may be the phased maternal chromosome set and phased paternal chromosome set.

### Simulated Parental Gamete Generation

Once the phased maternal chromosome set and phased paternal chromosome are generated, a meiotic recombination model may be used to generate a maternal gamete based on the phased maternal chromosome set and a paternal gamete based on the phased paternal chromosome set. Furthermore, the meiotic recombination model may generate a maternal gamete and paternal gamete based on one or more meiotic recombination sites of interest.

In some embodiments, a maternal gamete and paternal gamete may be simulated using software-based approaches, such as by using a parental support model as described above in FIGS. 2A-2B and/or using one or more meiotic recombination models, which may be included in the parental support model. In some embodiments, meiotic recombination sites of interested (e.g., represented as breakpoints) may be derived using software based approaches. The respective phased parental chromosome set (e.g., maternal chromosome set or paternal chromosome set) is then intersected at the meiotic recombination sites of interest to generate the corresponding parental gamete (e.g., maternal gamete or paternal gamete).

### Simulated Embryo Genotype Generation

Once the maternal gamete and paternal gamete are generated, these gametes may be combined to generate a simulated embryo genotype. As mentioned above, the above operations may be repeated a desired number of times such that one or more simulated embryo genotypes may be generated for a prospective embryo. In some embodiments, a threshold number of simulated embryo genotypes may be required to increase confidence in downstream disease probability determinations. For instance, in some embodiments, at least ten or more simulated embryo genotypes may be required. A PRS may then be generated for each simulated embryo genotype and the PRSs may be used to determine a probability of disease distribution for the prospective embryo as further described below.

### Polygenic Risk Score Determination

### Polygenic Risk Scoring

Once the one or more simulated embryo genotypes are generated as described above, a polygenic risk model may be applied to each simulated embryo genotype to generate a polygenic risk score (PRS), also known as a polygenic score (PGS) or genetic risk score (GRS), for the corresponding simulated embryo genotype. The one or more PRSs may be stored in a PRS set. A PRS may be indicative of the risk of a specific condition for an embryo with the genetic makeup of the simulated embryo genotype. The PRS determines whether disease causing variants are present or absent in the simulated embryo genotype (as inherited from an antecedent genome). The presence or absence of certain disease causing variants may increase disease susceptibility. Disease causing variants may include, for example, single nucleotide variants (SNVs), small DNA base insertions or deletions (indels), and/or copy number variants (CNVs).

In particular, the polygenic risk model may generate a polygenic risk score for a simulated embryo genotype using equation 3 described below.

$$PRS = {\sum }_{i = 1}^{n} {B}_{i} {x}_{i} \qquad ( 3 )$$

In equation 3, βᵢ is the log odds ratio for an associated allele for a SNP i, xᵢ is the allele dosage for SNP i, and n is the total number of SNPs included in the polygenic risk model.

Table 4 depicts example log odds ratios associated with various disease causing variants used to calculate a vitiligo PRS.

TABLE 4

| | Risk | | | Risk | |
|---|---|---|---|---|---|
| Variant ID | Allele | β | Variant ID | Allele | β |
| chr2_55845109_T_C_b37 | C | 0.392 | chr1_8484823_A_G_b37 | A | 0.199 |
| chr2_163110536_A_G_b37 | G | 0.288 | chr1_172715702_A_G_b37 | G | 0.285 |
| chr2_204740675_G_A_b37 | A | 0.166 | chr11_35274829_A_G_b37 | G | 0.215 |
| chr2_242407588_G_A_b37 | G | 0.223 | chr11_95320808_T_C_b37 | C | 0.322 |
| chr2_112010486_T_C_b37 | C | 0.157 | chr11_89017961_G_A_b37 | G | 0.4 |
| chr16_90026512_T_C_b37 | T | 0.315 | chr11_64021605_G_A_b37 | G | 0.139 |
| chr9_127071493_T_C_b37 | C | 0.148 | chr7_29132279_A_G_b37 | G | 0.668 |
| chr22_37581485_C_A_b37 | A | 0.293 | chr4_102223386_C_A_b37 | C | 0.151 |
| chr22_41767486_T_C_b37 | T | 0.248 | chr15_28535266_A_C_b37 | A | 0.358 |
| chr17_40289012_G_A_b37 | A | 0.166 | chr21_43851828_T_A_b37 | A | 0.358 |
| chr3_188089254_C_T_b37 | T | 0.278 | chr14_25102160_C_T_b37 | C | 0.207 |
| chr3_71557945_C_A_b37 | C | 0.223 | chr6_396321_C_T_b37 | C | 0.261 |
| chr3_196347253_T_A_b37 | T | 0.151 | chr6_167394634_A_T_b37 | A | 0.236 |
| chr19_50168871_G_A_b37 | G | 0.198 | chr6_90976768_G_A_b37 | A | 0.247 |
| chr19_4831878_A_G_b37 | A | 0.174 | chr6_2908591_A_G_b37 | A | 0.236 |
| chr8_129012574_C_T_b37 | T | 0.166 | chr6_32591291_T_A_b37 | A | 0.571 |
| chr8_133931055_A_T_b37 | A | 0.191 | chr13_43070206_T_G_b37 | G | 0.148 |
| chr20_49123043_A_G_b37 | G | 0.148 | chr10_115488331_G_A_b37 | G | 0.186 |
| chr20_32665748_A_G_b37 | G | 0.462 | chr10_6098824_T_C_b37 | T | 0.301 |
| chr1_198672299_T_C_b37 | T | 0.198 | chr10_63779871_C_T_b37 | T | 0.166 |
| chr1_114377568_A_G_b37 | A | 0.329 | chr18_60028655_A_C_b37 | C | 0.215 |

### Normalization

In some embodiments, each PRS may be normalized using one or more normalization methods. In some embodiments, each PRS is normalized based on population data. In some embodiments, the population data may be ancestry specific population data. Ancestry specific population data may be population data collected for a specific ancestry. In some embodiments, one or more haplotypes of a simulated embryo genotype may be evaluated to identify corresponding ancestry for each haplotype. The ancestry with the largest portion (e.g., the largest percentage) may be selected for the simulated embryo genotype and ancestry specific population data corresponding to that ancestry may be selected for the simulated embryo genotype. As such, each simulated embryo genotype may be normalized using ancestry aware data.

One example normalization method is standard score normalization, which may be represented in equation 4.

$$z = \frac{x - μ}{σ} \qquad ( 4 )$$

In equation 4, z is the normalized PRS, x is the raw PRS (as determined using equation 1), μ is the mean for a matching population, and σ is the standard deviation for the matching population.

Additionally, or alternatively, a PRS may be normalized by centering the PRS and dividing the centered PRS by the standard deviation as depicted below in equation 5.

$$z = \frac{{PRS}_{centered}}{σ} \qquad ( 5 )$$

In equation 5, z is the normalized PRS, PRS_{centered} is the centered PRS, and σ is the standard deviation of a population most closely related to the simulated embryo genotype, such as a population described in the 1000 Genomes Project. The centered PRS value may be determined by subtracting out the PRS value predicted from a linear regression of PRS against the first four principal component (PCs) score in control individuals (e.g., individuals without the phenotype of interest), as shown in equations 6 and 7.

$${PRS}_{centered} = x - {x}_{pred} \qquad ( 6 )$$

$${x}_{pred} = {\sum }_{i = 1}^{4} {{β}_{i} ( PC )}_{i} \qquad ( 7 )$$

In equation 7, βᵢ is the log odds ratio for an associated allele for a SNP i and (PC)ᵢ is the corresponding principal component score as determined using a linear regression. In equation 6, x is the PRS value and x_{pred} is the predicted PRS value.

### Probability of Disease Distribution Determination

After determining the one or more PRSs (and in some embodiments, after normalization), each PRS for a simulated embryo genotype may be used to determine a probability of disease distribution for a prospective embryo. In some embodiments, in order to determine an accurate probability of disease distribution, a threshold number of simulated embryo genotypes may be required. In some embodiments, at least ten or more simulated embryo genotypes may be required.

Additionally, one or more risk thresholds may be determined for each disease of interest. In some embodiments, a risk threshold may be a PRS value (or relative risk value as further discussed below) which is associated with a higher than average risk for the disease. Risk thresholds may be determined using clinical data or other data.

### Conversion to Relative Risk

After determining the one or more PRSs and after normalization, each PRS may be converted to a relative risk (RR) of disease. The RR may be determined using an effect size model. The effect size model may receive each PRS and determine a corresponding odds ratio for the PRS according to equation 8.

$${OR}_{PRS} = {e}^{{B}_{PRS * zscore}} \qquad ( 8 )$$

In equation 8, zscore is the normalized PRS as described above and B_{PRS} is the log odds ratio for the PRS. The effect size model may then determine the RR according to equation 9.

$$RR = \frac{{OR}_{PRS}}{( 1 - prev ) + ( prev * {OR}_{PRS} )} \qquad ( 9 )$$

In equation 9, prev is the prevalence of the disease. Once the PRSs are converted into RRs, the probability of disease distribution may be represented using RRs instead of PRSs.

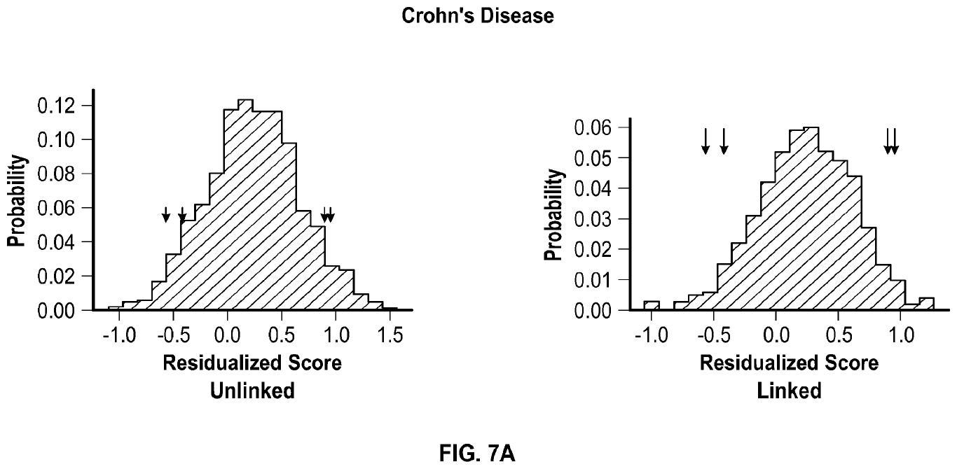

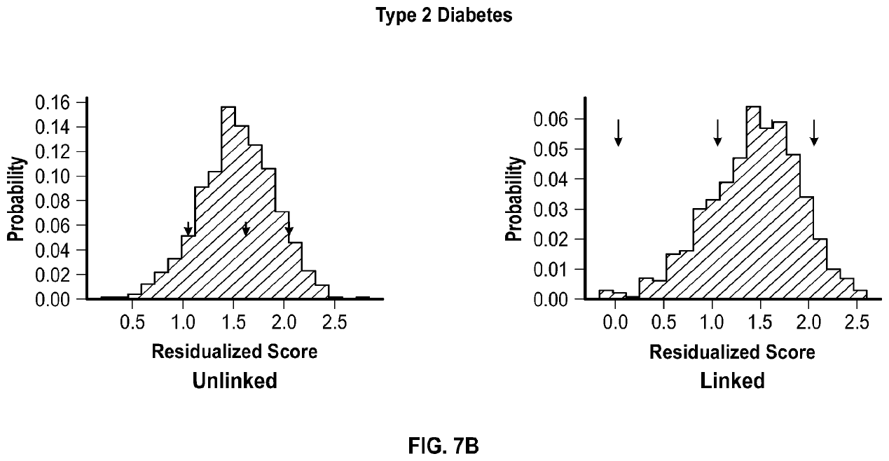

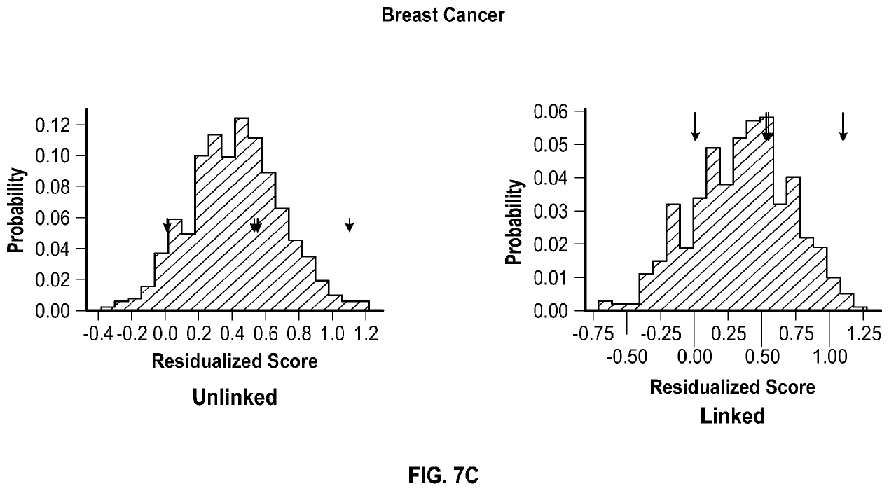

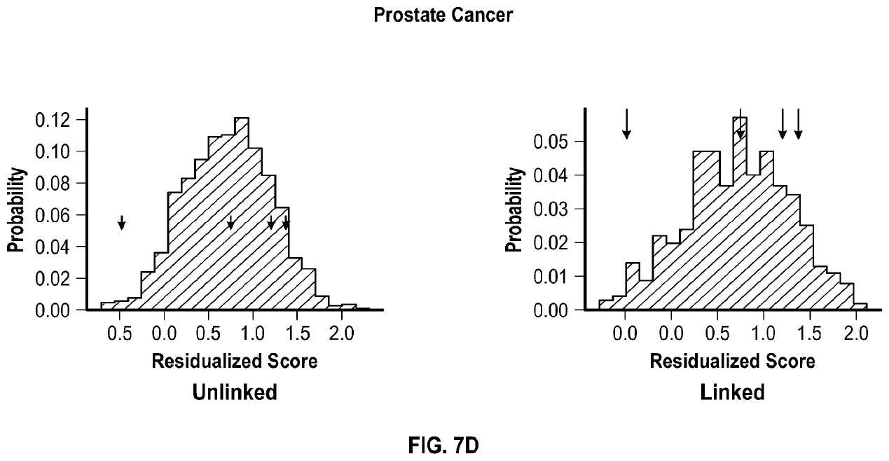

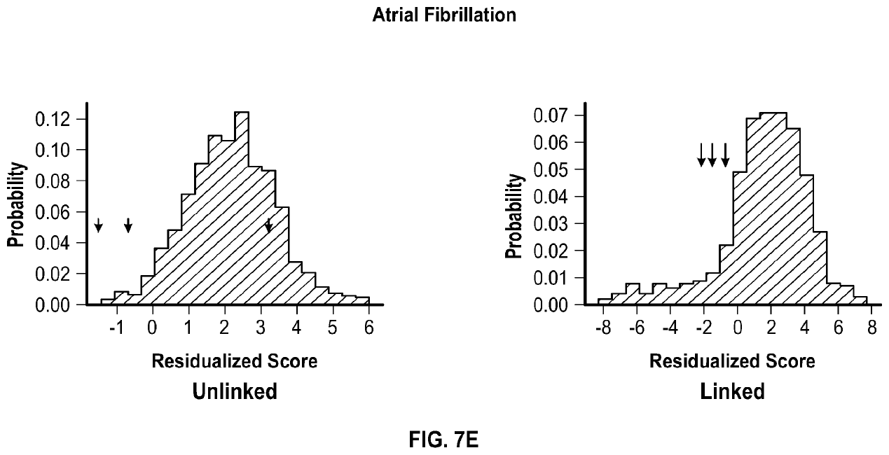

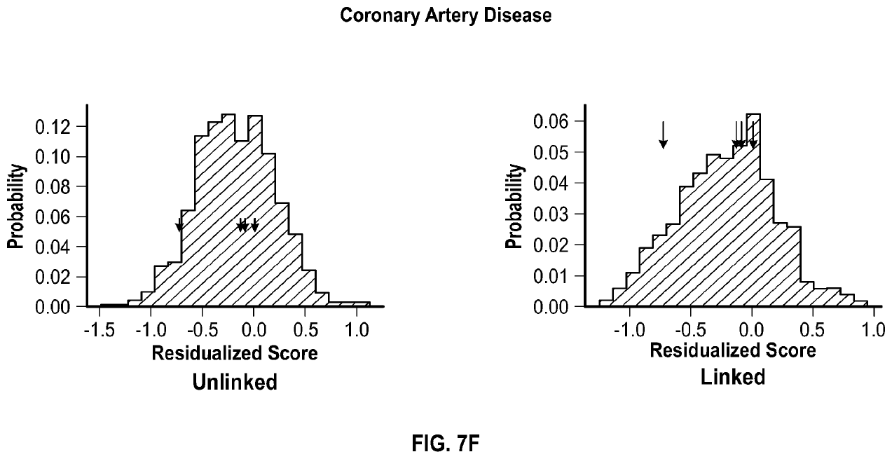

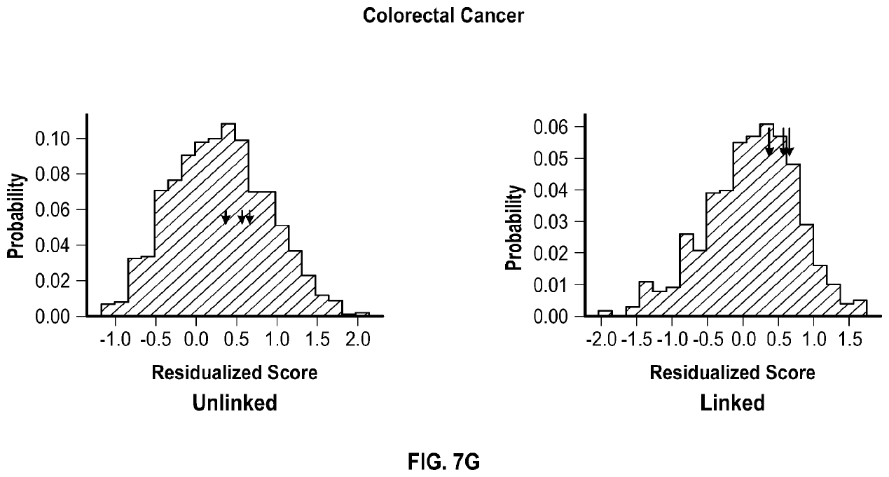

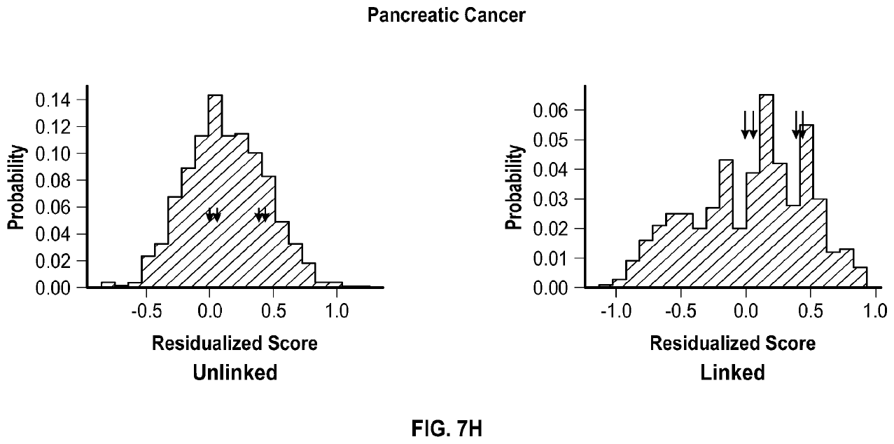

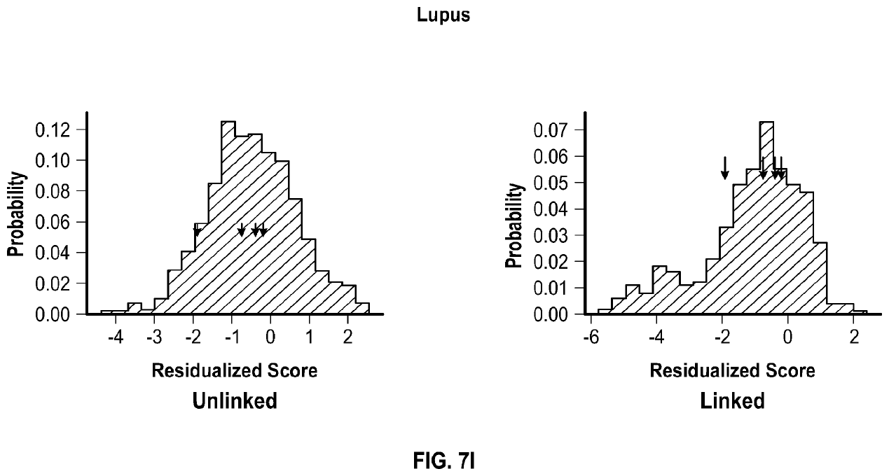

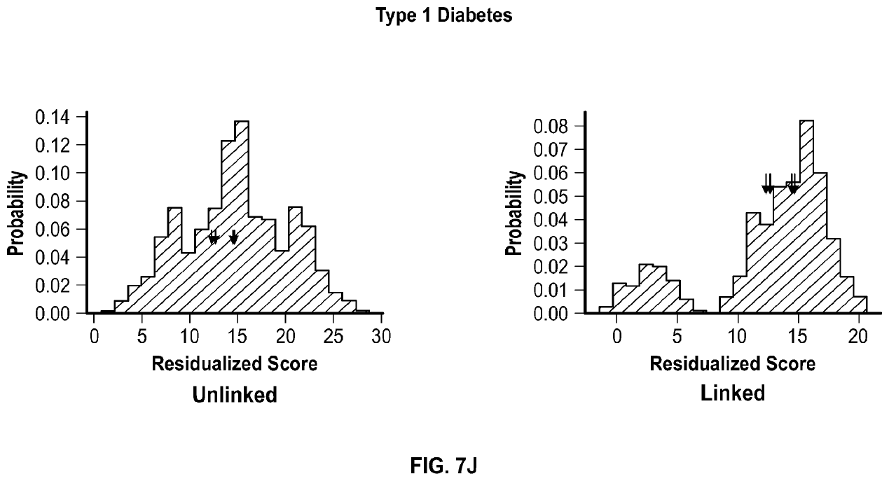

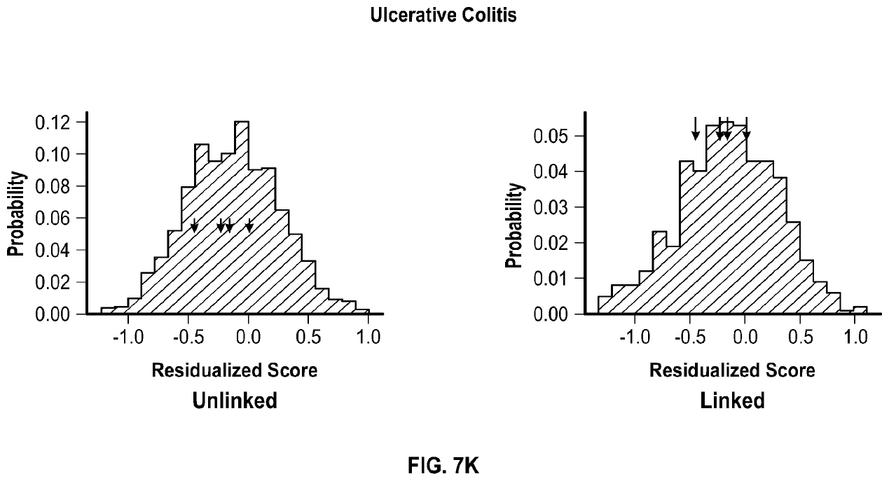

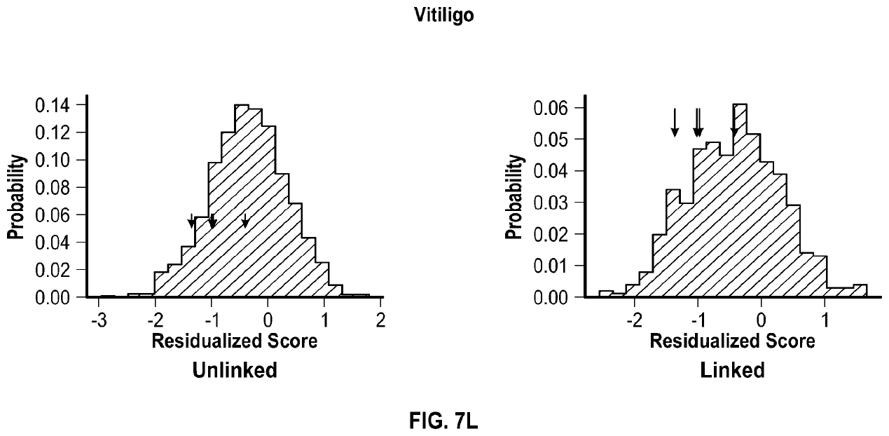

FIGS. 7A-7K depict additional examples of example probability of disease distributions using RR for various diseases. Furthermore, in FIGS. 7A-7K both an unlinked approach method and linked approach method are used to generate the probability of disease distributions. Similarly here, the arrows represent predicted risk of the respective disease as determined for actual embryos. As depicted in FIGS. 7A-7K, in some instances, the unlinked approach approximates the probability of disease distribution fairly closely to the linked approach, such as in FIG. 7A which depicts the probability of disease distributions for Crohn's disease. However, in many other cases, the probability of disease distributions as determined by the unlinked approach significantly diverges from the probability of disease distributions determined by the linked approach, such as in FIG. 7J, which depicts the probability of disease distribution for type 1 diabetes. As described above, this divergence is due to the failure of the unlinked approach to consider risk-contributing variants that are linked on the same haplotype that are transmitted together and thus coordinately increase risk.

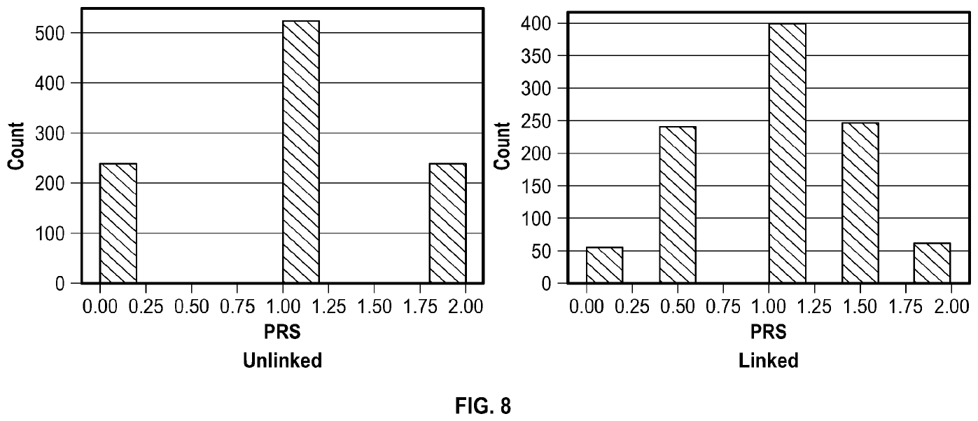

FIG. 8 depicts an example score distribution for the unlinked approximation and linked approximation. To better illustrate the effect of the linked approximation on PRS determination, a simplified model with two sites may be considered. Each parent may be heterozygous at both sites (0/1). In the unlinked approximation, the probability that the child has genotype 0/0, 0/1, and 1/1 is 0.25, 0.5, and 0.25, respectively. It can be assumed the weight of each risk allele is 0.5 to obtain the unlinked score distribution depicted in FIG. 8. In the linked approach, it is assumed these two sites are linked and can be collapsed into a single site, where the weight of the risk allele is 1 to obtain the linked score distribution depicted in FIG. 8. As shown in FIG. 8, the mean PRS may be the same but the distribution of PRS changes when linkage is considered.

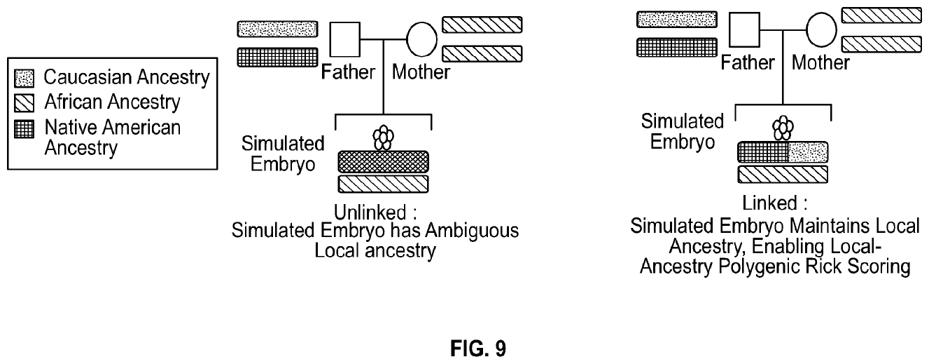

Additionally, FIG. 9 further illustrates the impact of an unlinked approach and linked approach on a simulated embryo genotype with respect to the transmission of contextual ancestry information. In the unlinked approach, the paternal contribution to the simulated embryo genotype is ambiguous and thereby may result in artifactual shifts in PRS-predicted risk. Conversely, with the linked approach, local ancestry is maintained, thus allowing for PRS models to consider local ancestry approaches when determining risk scoring.

### Disease Occurrence Risk

Once a probability of disease distribution is determined for the prospective embryo, an occurrence risk for one or more diseases may also be determined for the prospective embryo. The occurrence risk may be determined based on the probability of disease distribution and one or more thresholds. The one or more thresholds may be one or more PRS thresholds and/or RR thresholds which delineate PRSs associated with a high risk of a disease. The percentage of simulated embryo genotypes which satisfy the threshold (e.g., are above the threshold) may be used to determine the occurrence risk for the prospective embryo. The occurrence risk may be indicative of a likelihood for a particular disease to occur in the prospective embryo based on the simulated embryo genotypes determined using the linked approximation.

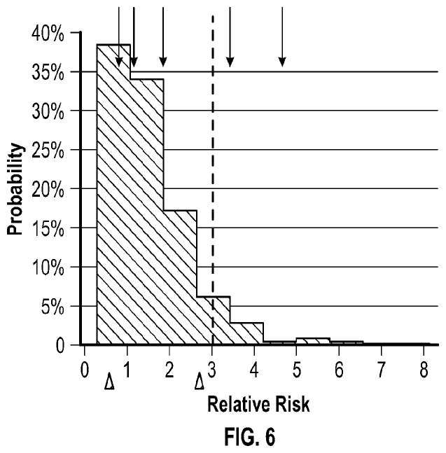

For example, FIG. 6 depicts an example probability of disease distribution using RR for vitiligo. As shown in FIG. 6, a disease risk distribution (e.g., vitiligo) for a prospective embryo as determined using simulated embryo genotypes generated and processed as described above. FIG. 6 further depicts the triangles, which represent calculated parent RR based on provided and sequenced samples. Additionally, the arrows represent the predicted risk of vitiligo for actual embryos. The dotted line is a threshold used to delineate PRSs associated with a high risk of disease. The resulting probability of disease distribution shown in FIG. 6 suggest that 93% of embryos would have a RR below the threshold RR value of 3 and that 7% of embryos would have a RR at or above the threshold value. As such, the occurrence risk for the prospective embryo may be 7%. As such, families, medical providers, and other parties may be informed that there is a relatively low risk for a prospective embryo to have a genotype associated with a high risk of vitiligo.

### EXAMPLE IMPLEMENTATIONS

One example implantation of the linked approximation is within a clinical setting. In particular, a clinical setting which performs pre-implantation genetic testing for polygenic disorders (PGT-P). Typically, women who undergo IVF often have more embryos available for implantation than needed. This gives them the opportunity not only to maximize the chance of a successful pregnancy, but also to minimize the chance of passing on a disease that affects the mother or any of her family members. Predicting embryo disease risk is possible for any disease which has a genetic component, which includes the majority of common and rare diseases.

Pre-implantation genetic testing is already routinely performed for aneuploidy screening (PGT-A), which involves obtaining embryo biopsies. The embryonal cells gathered in this process can then be genotyped through sequencing or microarray technologies to collect the base-pair level information that is needed to predict common disease risk (PGT-P) for the particular embryo. Based on these predictions, the IVF clinic is then able to choose an embryo for implantation which does not carry an elevated disease risk.

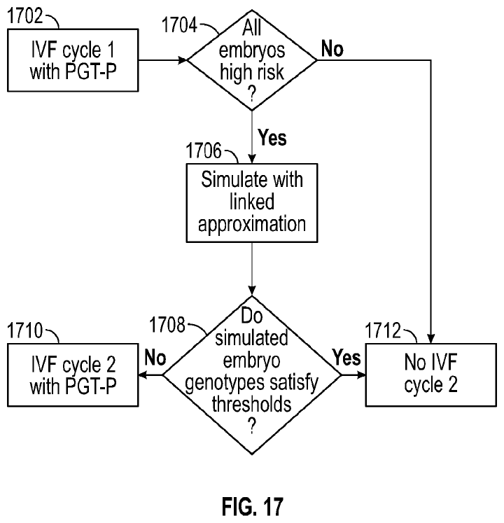

However, in some instances, a particular round of IVF may yield embryos which are all determined have a high risk of disease. As depicted in the example flowchart of FIG. 17, a first IVF cycle (e.g., cycle 1) may be performed for a couple PGT-P may be used to infer the risk of disease for each embryo as shown in operation 1702. At operation 1704, it may be determined whether all the embryos are high risk for one or more diseases based on the PGT-P results. If one or more of the embryos are determined to not be high risk for one or more diseases, those embryos may be chosen for implantation and no additional cycles of IVF are needed such that the process may proceed to operation 1712.

If all of the embryos are high risk, the process may proceed to operation 1706, where a prospective embryo may be simulated using the linked approach as described above. At operation 1708, it may be determined whether an occurrence risk for the prospective embryo satisfy one or more thresholds. For example, a threshold of 50% may be set such that an occurrence risk with a value at or below 50% satisfies the threshold. If an occurrence risk above 50% is determined for the prospective embryo, the threshold is not satisfied.

If the occurrence risk does not satisfy the one or more thresholds, the process proceeds to operation 1712. At operation 1710, an additional round of IVF (e.g., cycle 2) may not be recommended. This recommendation may occur when there is little chance of success of a prospective embryo not having a high risk of disease (e.g., as determined from PGT-P).

If the occurrence risk satisfies the one or more thresholds, the process proceeds to operation 1710. At operation 1710, it may be determined that a second cycle (e.g., cycle 2) of IVF with PGT-P is recommended.

Regardless of the outcome, either the recommendation for an additional round of IVF may be output to clinical practitioners (e.g., doctors, nurses, obstetricians, etc.), geneticists, patients, etc. such that the parties involved in determining a next course of action may be better informed of the risks and potential success rates of another round of IVF. The linked approach may be particularly beneficial when there are large differences in predicted risk among simulated embryo genotypes.

### Example Implementation System

The methods described herein may be implemented on a variety of systems. For instance, in some embodiments the system may be used for generating phased parental chromosome sets, determining recombination sites of interest, generating one or more simulated embryo genotypes, applying polygenic risk models to the one or more simulated embryo genotypes, determining a probability of disease distribution, etc.

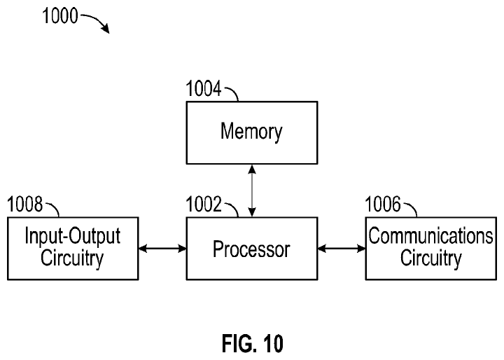

The system may include one or more system devices, which may be embodied by one or more computing devices or servers, shown as apparatus 1000 in FIG. 10. As illustrated in FIG. 10, the apparatus 1000 may include a processor 1002, memory 1004, and communications hardware 1006, each of which will be described in greater detail below. While the various components are only illustrated in FIG. 10 as being connected with apparatus 1000, it will be understood that the apparatus 1000 may further comprise a bus (not expressly shown in FIG. 10) for passing information amongst any combination of the various components of the apparatus 1000. The apparatus 1000 may be configured to execute various operations described above.

The processor 1002 (and/or co-processor or any other processor assisting or otherwise associated with the processor) may be in communication with the memory 1004 via a bus for passing information amongst components of the apparatus. The processor 1002 may be embodied in a number of different ways and may, for example, include one or more processing devices configured to perform independently. Furthermore, the processor may include one or more processors configured in tandem via a bus to enable independent execution of software instructions, pipelining, and/or multithreading. The use of the term “processor” may be understood to include a single core processor, a multi-core processor, multiple processors of the apparatus 1000, remote or “cloud” processors, or any combination thereof.

The processor 1002 may be configured to execute software instructions stored in the memory 1004 or otherwise accessible to the processor (e.g., software instructions stored on a separate storage device). In some cases, the processor may be configured to execute hard-coded functionality. As such, whether configured by hardware or software methods, or by a combination of hardware with software, the processor 1002 represents an entity (e.g., physically embodied in circuitry) capable of performing operations according to various embodiments of the present invention while configured accordingly. Alternatively, as another example, when the processor 1002 is embodied as an executor of software instructions, the software instructions may specifically configure the processor 1002 to perform the algorithms and/or operations described herein when the software instructions are executed.

The memory 1004 is non-transitory and may include, for example, one or more volatile and/or non-volatile memories. In other words, for example, the memory 1004 may be an electronic storage device (e.g., a computer readable storage medium). The memory 1004 may be configured to store information, data, content, applications, software instructions, or the like, for enabling the apparatus to carry out various functions in accordance with example embodiments contemplated herein.

The communications hardware 1006 may be any means such as a device or circuitry embodied in either hardware or a combination of hardware and software that is configured to receive and/or transmit data from/to a network and/or any other device, circuitry, or module in communication with the apparatus 1000. In this regard, the communications hardware 1006 may include, for example, a network interface for enabling communications with a wired or wireless communication network. For example, the communications hardware 1006 may include one or more network interface cards, antennas, buses, switches, routers, modems, and supporting hardware and/or software, or any other device suitable for enabling communications via a network. Furthermore, the communications hardware 1006 may include the processing circuitry for causing transmission of such signals to a network or for handling receipt of signals received from a network.

The communications hardware 1006 may be configured to provide output to a user and, in some embodiments, to receive an indication of user input. The communications hardware 1006 comprise a user interface, such as a display, and may further comprise the components that govern use of the user interface, such as a web browser, mobile application, dedicated user device, or the like. In some embodiments, the communications hardware 1006 may include a keyboard, a mouse, a touch screen, touch areas, soft keys, a microphone, a speaker, and/or other input/output mechanisms. The communications hardware 1006 may utilize the processor 1002 to control one or more functions of one or more of these user interface elements through software instructions (e.g., application software and/or system software, such as firmware) stored on a memory (e.g., memory 1004) accessible to the processor 1002.

### EXAMPLES

### Example 1

Preimplantation genetic testing (PGT) of in vitro fertilized embryos was performed to infer an inherited genome sequence in 110 embryos across 10 couples and model susceptibility across 12 common conditions. The prospective simulated embryo was then compared to the genome sequence of the born child and also used to predict common disease risk through calculating polygenic risk scores and inferring the inheritance of rare variants with high impact on disease risk.

Table 5 describes a summary of each couple (each assigned to a respective case identifier). Performance for each case was determined by comparing genotypes from a simulated embryo genotype with the born child's DNA genotype. As shown in Table 5, accuracies ranging from 99.0-99.4% were obtained at sites used in polygenic prediction in Day-5 embryos and 97.2-99.1% in Day-3 embryos. Case 1 includes only Day-3 embryos and case 2 includes both Day-3 and Day-5 embryos. All other cases included Day-5 embryos only. Statistics are broken down by genotype (heterozygous or homozygous) in the born child. PGT from embryo biopsies were performed by a commercial lab (e.g., Natera, formerly Gene Security Network) on the HumanCytoSNP-12 BeadChip array, ranging from 3 to 33 embryos. The coverage and accuracy were assessed at genomic positions that are high-confidence genotype calls in parents and born child.

TABLE 5

| | | | | # of | | | | | | |
|---|---|---|---|---|---|---|---|---|---|---|
| | WGS | | | Embryos | | | Total | Total # | | |
| | Library | | Born | biopsied | | Reason | high- | sites | High- | # PRS sites |
| Case | Preparation | Day of | Child | Total | | for | confidence | predicted | confidence | predicted |
| ID | Technique | Biopsy | sequenced | (euploid) | | IVF | sites | (%) | PRS sites | (%) |
| 6 | PCR + | 5 | Yes | 5 | (3) | PGT- | 6798513 | 6288512 | 12534 | 12514 |
| | WGS | | | | | A | | (92.50%) | | (99.84%) |
| 7 | PCR + | 5 | Yes | 3 | (3) | PGT- | 5796784 | 5411037 | 12799 | 12768 |
| | WGS | | | | | A | | (93.35%) | | (99.76%) |
| 3 | PCR + | 5 | Yes | 12 | (8) | PGT- | 6049554 | 5682135 | 12844 | 12821 |
| | WGS | | | | | M | | (93.93%) | | (99.82%) |
| 2 | PCR + | 3, 5 | Yes | 5 | (2) | PGT- | 6853313 | 6398275 | 12877 | 12829 |
| | WGS | | | | | M | | (93.36%) | | (99.63%) |
| 1 | PCR + | 3 | Yes | 16 | (10) | PGT- | 6273898 | 5714910 | 12834 | 12660 |
| | WGS | | | | | M | | (91.09%) | | (98.64%) |
| 4 | PCR + | 5 | Yes | 5 | (4) | PGT- | 5828420 | 5446848 | 12695 | 12624 |
| | WGS | | | | | M | | (93.45%) | | (99.44%) |
| 5 | PCR free + | 5 | Yes (2) | 8 | (4) | PGT- | 6052527 | 5738390 | 12821 | 12800 |
| | WGS | | | | | A | 5985546 | (94.81%) | 12815 | (99.84%) |
| | | | | | | | | | | 12793 |
| | | | | | | | | | | (99.83%) |
| 8 | PCR free + | 5 | Yes | 8 | (8) | PGT- | 5815774 | 5675376 | 12845 | 12809 |
| | WGS | | | | | A | | (94.82%) | | (99.72%) |
| 9 | PCR free + | 5 | Yes | 15 | (5) | PGT- | 6276690 | 5484833 | 12844 | 12827 |
| | WGS | | | | | A | | (94.31%) | | (99.87%) |
| 10 | Tell-Seq | 5 | No | 33 | (21) | PGT- | N/A | 5932931 | N/A | N/A |
| | | | | | | M | | (94.52%) | | |

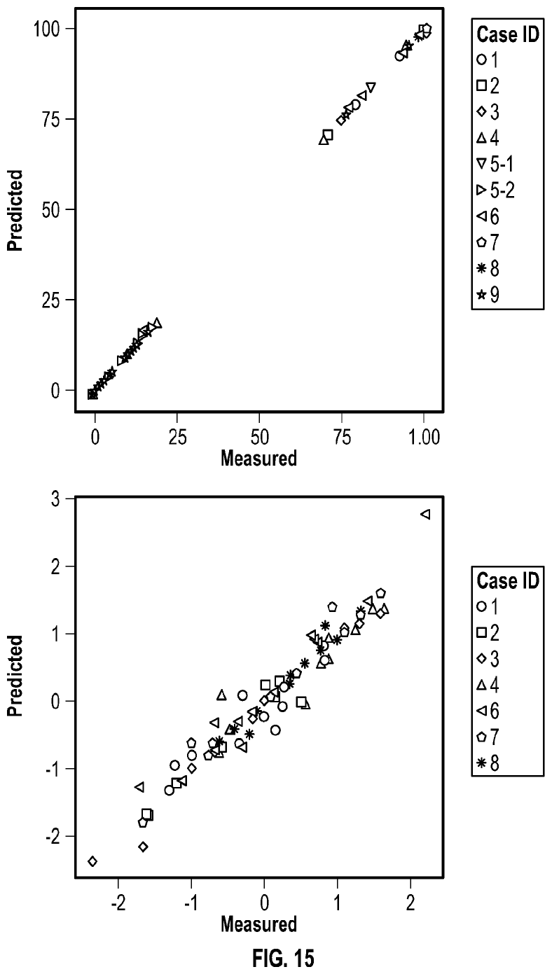

Additionally, FIG. 15 depicts the correlation of PRS from simulated embryo predictions and the born child. The first graph in FIG. 15 illustrates the close correlation between between predicted and measured (born child) raw PRS, consistent with genotype concordance between predicted and measured polygenic risk. The second graph in FIG. 15 depicts the correlation between predicted and measured z-score derived from raw PRS (r²=0.947). Families 5 and 9 were excluded from this analysis as the approach to mean-center polygenic risk using population ancestry is unable to account for admixture.

In four cases where fresh blood samples were available, synthetic long read sequencing was also performed on both the mother sample and father sample. Modifications to the above protocol included additionally performing high molecular weight DNA and library preparation using a TELL-Seq library using standard protocols, except for reduced transposable enzyme.

### Example 2

For whole genome sequencing of parental genotypes and born children genotypes, an average depth of 30x was targeted. Actual mean coverage for all samples ranged from between 29x and 111x. Table 6 depicts the actual mean depth used for each case for the corresponding mother, father, and child sequencing. The percentage greater than 20x (%≥20x) is indicative of the percentage of genomic bases covered by at least 20 sequence reads.

TABLE 6

| | Mother | | Father | | Child | |
|---|---|---|---|---|---|---|
| | Mean | | Mean | | Mean | |
| Case ID | Depth | % ≥20X | Depth | % ≥20X | Depth | % ≥20X |
| 1 | 38X | 97% | 42X | 96% | 38X | 95% |
| 2 | 38X | 96% | 43X | 96X | 43X | 97% |
| 3 | 43X | 98% | 41X | 95% | 41X | 97% |
| 4 | 34X | 92% | 32X | 85% | 29X | 83% |
| 5 | 36X | 96% | 31X | 90% | 40X | 97% |
| | | | | | 41X | 95% |
| 6 | 38X | 96% | 30X | 84% | 34X | 88% |
| 7 | 39X | 96% | 30X | 84% | 39X | 95% |
| 8 | 36X | 94% | 34X | 95% | 29X | 89% |
| 9 | 47X | 98X | 43X | 96% | 37X | 97% |
| 10 | 86X | 98% | 111X | 99% | N/A | N/A |

WGS primary analysis and secondary analysis were performed according to the Broad Institute's best practices pipeline (GATK), implemented by Sentieon Software. The human reference genome sequence (GRCh37) was mapped with Burrow-Wheeler Aligner (bwa) version 0.7.17. Genotyping for each parent and actual child was then performed using two steps. First, a joint variant calling on the parent and the born child captured sequences using Sentieon's GVCFtyper and filtered these based on internal quality control thresholds. Joint variant calling allows for all samples (e.g., the maternal sample, paternal sample, and born child sample) to be considered simultaneously to produce genotypes at many variant positions as opposed to variant positions detected from a given sample. The internal quality control thresholds may include a base quality control, a median depth (DP), Fisher Strand (FS), and a quality score normalized by allele depth (QD). These internal quality control thresholds may be used to identify sequencing errors. In particular, internal quality control thresholds were set as follows: BP greater than or equal to 20, DP greater than or equal to 8, FS less than 30, and QD greater than 4. Secondly, genotypes were called at sites specific to polygenic models with a read depth of at least 8x.

### Example 3

Embryo biopsies were genotyped by extracting and amplifying embryo DNA, followed by genotyping using a rapid SNP microarray protocol (e.g., on Illumina's HumanCytoSNP-12 BeadChip). Sibling embryos' and parents' SNP microarray measurements were combined using the parental support model to determine a maximum likelihood estimate (MLE) phase of heterozygous SNVs in each parent by combining recombination frequencies from a HapMap database with SNP array measurements from parents and SNP array measurements from sibling embryos. The combination may yield parental support haplotypes.

Secondly, the HMM of the parental support model was used to determine the most likely parental haplotype transmitted to each embryo given SNP array measurements from the embryo and MLE phase for each parent. The outputs of the HMM were used to inform the meiotic recombination sites.

### Example 4

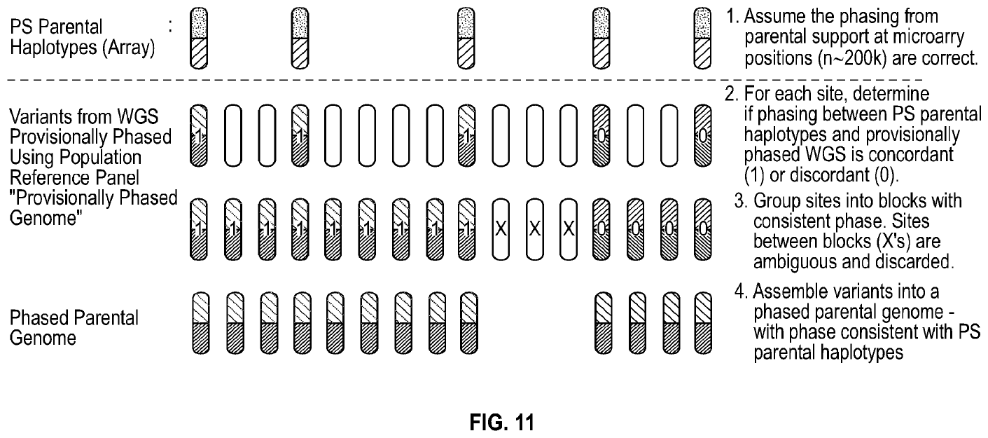

To phase WGS-derived variants in each parent, we another simulation model was used to estimate haplotypes for the parents (e.g., using SHAPEIT4). Default parameters were used with additional database data, such as available in the UK10K Imputation Cohort+1000 Genomes Phase 3 (data set EGAD00001000776), which served as a reference panel and parental support haplotype scaffold. This scaffold, consisting of ˜200,000 phased variants, serves to anchor phasing performed using the reference panel. FIG. 11 depicts the process of obtaining a phased parental genotype. Each chromosome may be processed independently and in parallel and all chromosomes are combined thereafter. Multi-allelic sites were excluded and discarded. To gain additional performance for rare variants not represented by reference panels, linked read sequencing of high molecular weight DNA may be used.

In particular, linked read sequencing data was generated for case IDs 5, 8, 9, and 10 using the TELL-Seq library preparation method. After read alignment and variant calling using the same process described above with the addition of maintaining molecular barcode information for each read, the molecular phase was inferred using another model (e.g., a HapCut2 model). The HapCut2 model is a maximum-likelihood-based tool for assembling haplotypes from DNA sequence reads. Positions of these haplotypes may be annotated with their global allele frequency using the gnomad database.

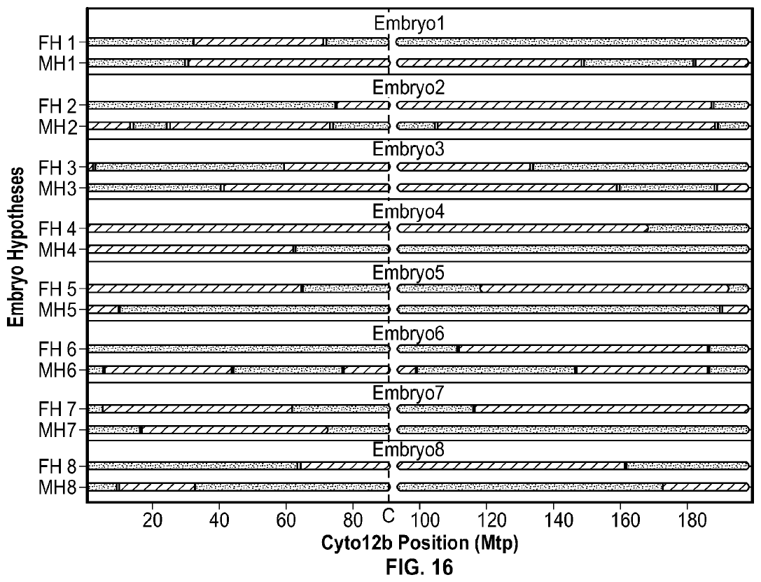

FIG. 16 depicts a plot of transmitted haplotypes on chromosomes 3 to 8 for sibling embryos derived from family 5. Transmitted haplotypes were output from parental support and form the basis of the PS Embryo Genotypes at microarray sites. Green and red lines denote parental haplotype 1 and 2 respectively for mother (MH) and father (FH) haplotypes in each embryo. (Regions of some uncertainty are colored yellow).

### Example 5

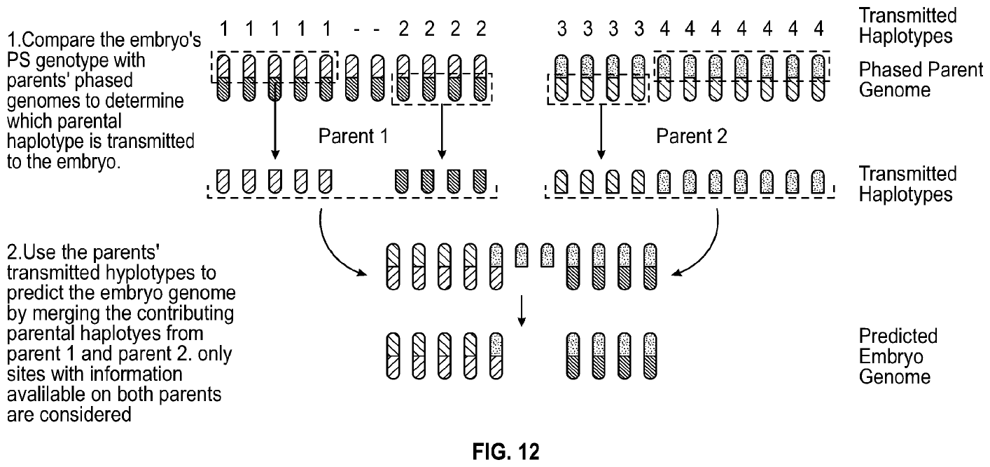

To predict the whole genome sequence of each sibling embryo genotypes were combined with phased parental genomes with the addition of chromosome-spanning haplotypes using the HMM of the parental support model. The parents' transmitted haplotype to the embryo was obtained by comparing the haplotypes and the sibling embryo's genotypes. This was performed process across each maternal and paternal chromosome. FIG. 11 and FIG. 12 depict this process in greater detail.

Low quality sites in parental and born child genomes may be filtered as well as multi-allelic sites and sites corresponding to a Mendelian error in the sequence data from each family to form a set of “high confidence sites” that were used to assess coverage and accuracy. Predicted embryo genotype calls (derived from reconstruction) are compared with variants called by sequencing of the born child's DNA.

High-confidence sites were annotated with population allele frequencies from the gnomAD v2.1 data set, which is comprised of approximately 15,000 whole genomes and 125,000 exomes derived from seven populations: African, Latino, Ashkenazi Jewish, East Asian, European, South Asian, and Other. Variants with an allele frequency <0.1% or not present in the gnomAD database were considered rare.

Table 7 depicts the accuracy of sites as predicted by a reference panel and using linked read sequencing.

TABLE 7

| | Parental | Total # | # rare | % rare | % |
|---|---|---|---|---|---|
| Case | Phasing | rare | sites | sites | Accuracy |
| ID | Method | sites | predicted | predicted | of sites |
| 5-1 | Reference panel | 175,150 | 82,194 | 46.93% | 91.18% |
| 5-1 | Linked reads | | 150,270 | 85.79% | 97.25% |
| 5-2 | Reference panel | 174,924 | 82,069 | 46.92% | 91.01% |
| 5-2 | Linked reads | | 150,083 | 85.80% | 97.20% |
| 8 | Reference panel | 96,284 | 54,043 | 56.13% | 93.53% |
| 8 | Linked reads | | 77,430 | 80.42% | 96.78% |
| 9 | Reference panel | 141, 682 | 72,452 | 51.14% | 92.13% |
| 9 | Linked reads | | 119,541 | 84.3% | 96.15% |

### Example 6

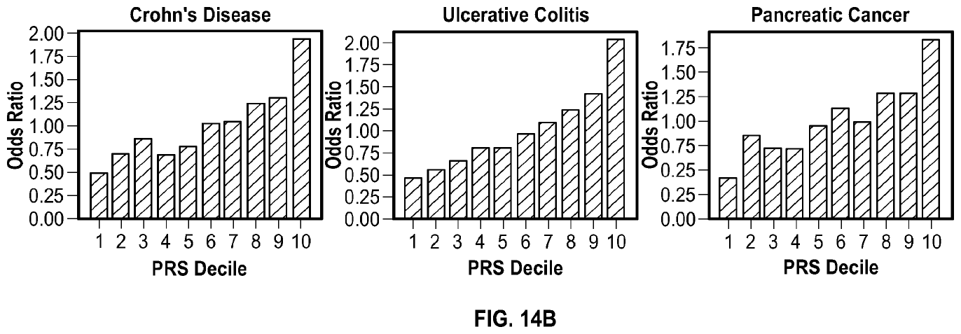

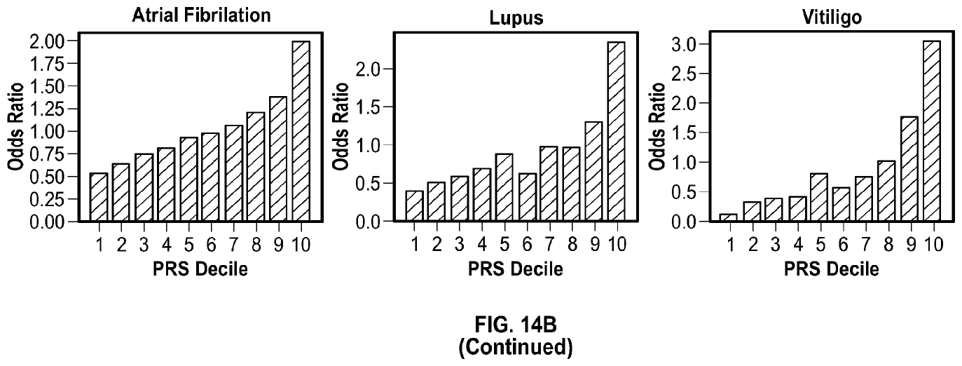

Polygenic risk scores and ancestral principal components were calculated using a similar approach for each simulated embryo genotype. In some instances, embryo genotype predictions were unable to be determined and thus the population allele frequency was used to adjust the PRS score. The PRS score was centered and standardized as described above and transformed into an odds ratio of disease given the PRS. Specifically, equation 3 was used, where Bis the PRS effect size (i.e. log odds per standard deviation) derived from the UK Biobank and PRS is the centered and standardized PRS. FIGS. 14A-14B illustrate disease odds ration by polygenic risk score decile.

### Example 7

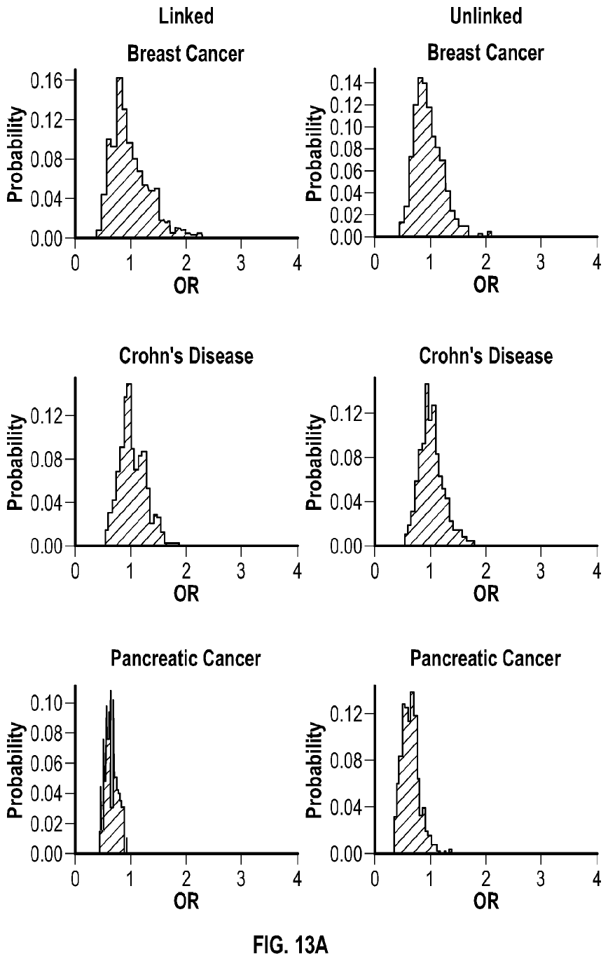

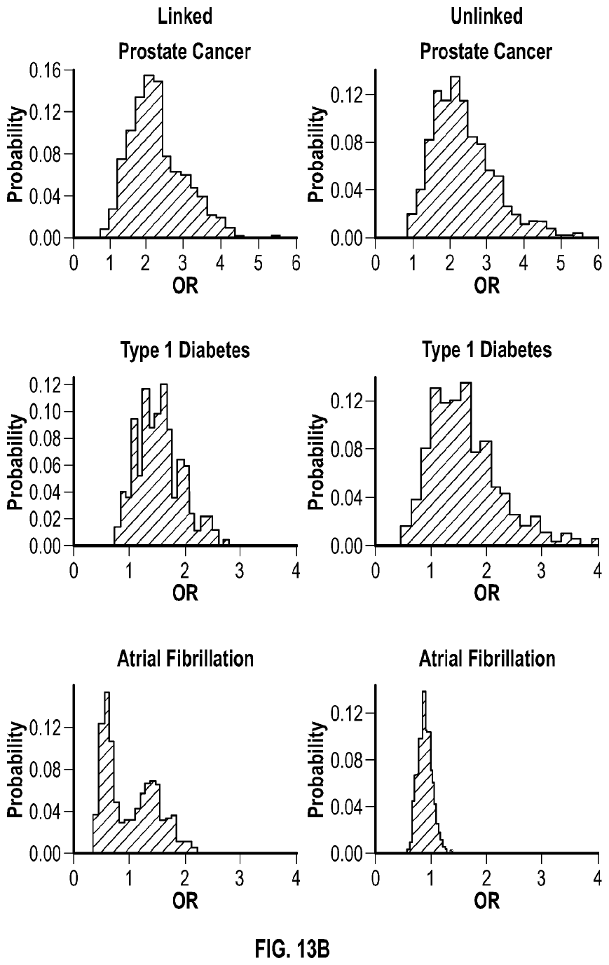

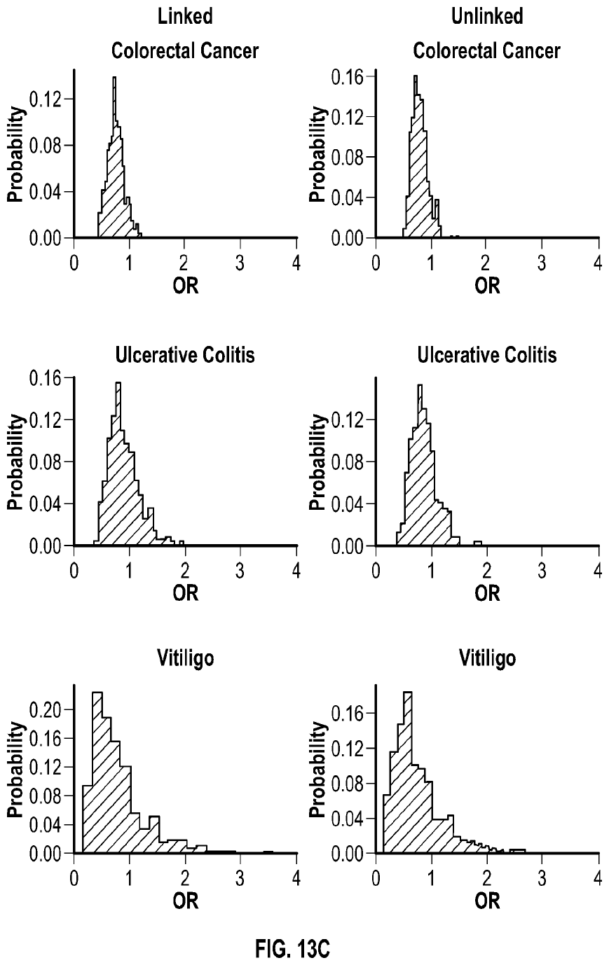

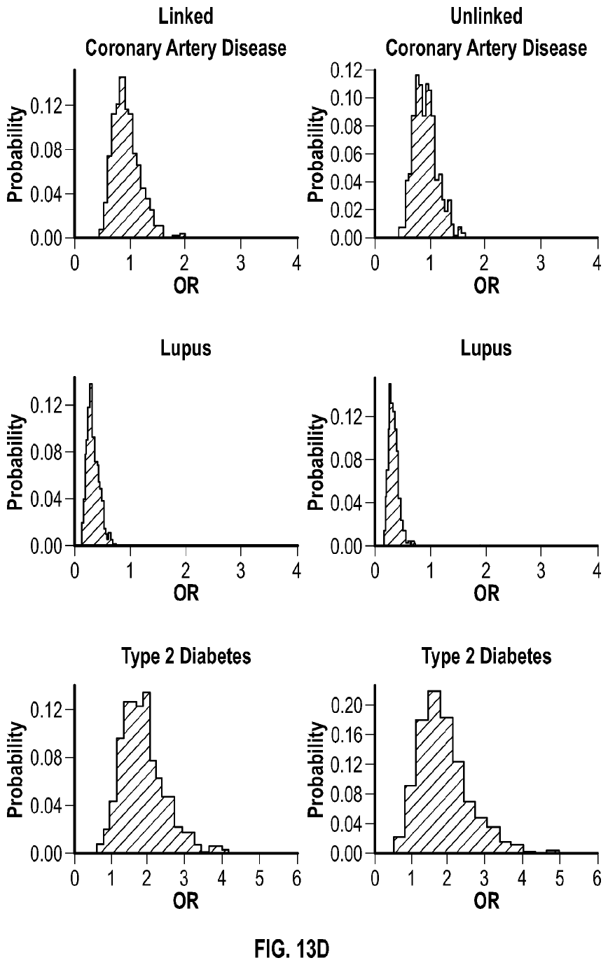

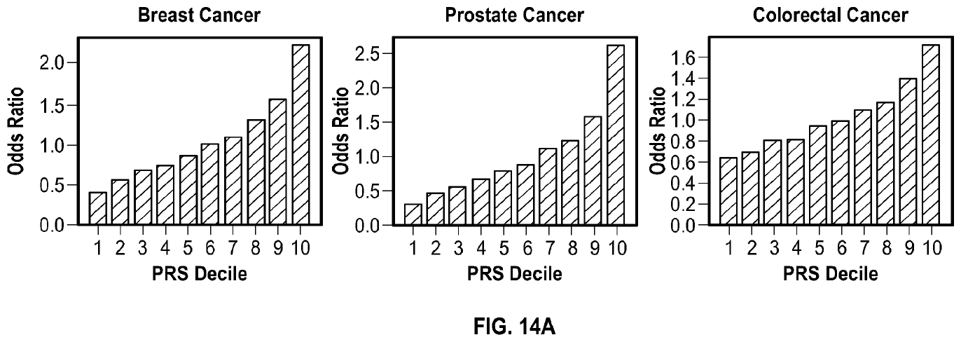

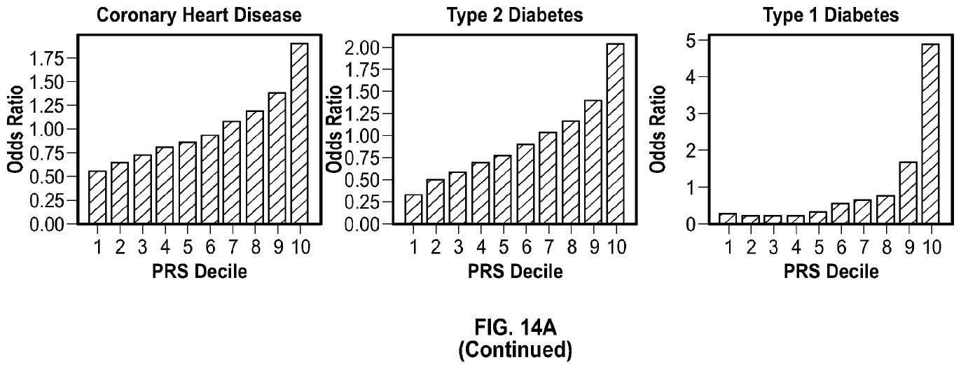

The linked approach was used to generate simulated embryo genotypes by starting with phased genomes of both parents, adding recombination's between the two mother or two father chromosomes (to approximate meiotic recombination in gametes), and combining these “virtual gametes” at random. Haplotypes derived using a parental support model were combined with with whole genome sequencing to generate phased parental genomes. A meiotic recombination model (e.g., ped-sim with a pedigree (two parents and one child) and a genetic map) was used to simulate sites for recombination. Breakpoints (e.g., meiotic recombination sites) derived from the meiotic recombination model were intersected with the phased parental genomes to generate “virtual gametes”. Virtual gametes from the mother and father were then combined to generate a simulated embryo genotype. PRS was performed in these simulated embryo genotypes as discussed above. To generate a distribution of risk scores, this process was repeated 500 times for each couple. In an unlinked approach, a simulated embryo genotype was generated by choosing one allele from each parent at random and make no assumptions on whether neighboring variants were linked. FIGS. 13A-13D depict the distribution of risk scores for various diseases using both the unlinked approach and the linked approach.

Furthermore, as depicted in Table 8, clinical PGT for aneuploidy (PGT-A) performed on the simulated embryo genotypes revealed 69 of the 110 embryos were euploid and 41 of 110 embryos were aneuploid. Whole genome reconstruction of embryos was achieved by performing high-coverage genome sequencing of both parents and array measurements of sibling embryos as described above.

TABLE 8

| | Predicted | Born Child | | Genotype |
|---|---|---|---|---|
| | chromosome | chromosome | Confirmed | Concordance |
| Case ID | count | count | status | at Array Sites |
| 1 | 46, XY | 46, XY | Normal | 99.33% |
| 2 | 46, XY | 46, XY | Normal | 99.86% |
| 3 | 46, XX | 46, XX | Normal | 99.81% |
| 4 | 46, XY | 46, XY | Normal | 99.57% |
| 5-1 | 46, XX | 46, XX | Normal | 99.91% |
| 5-2 | 46, XY | 46, XY | Normal | 99.91% |
| 6 | 46, XX | 46, XX | Normal | 99.74% |
| 7 | 46, XX | 46, XX | Normal | 99.88% |
| 8 | 46, XX | 46, XX | Normal | 99.76% |
| 9 | 46, XX | 46, XX | Normal | 99.85% |

### CONCLUSION

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

What is claimed is:

1. A method for determining a probability of disease distribution associated with a prospective embryo, the method comprising:
generating a phased maternal chromosome set and a phased paternal chromosome set;
determining one or more meiotic recombination sites of interest;
generating one or more simulated embryo genotypes based on the phased maternal chromosome set, the phased paternal chromosome set, and the one or more meiotic recombination sites of interest;
applying at least one polygenic risk model to the one or more simulated embryo genotypes to generate a polygenic risk score set, wherein the polygenic risk score set includes a polygenic risk score for each simulated embryo genotype of the one or more simulated embryo genotypes; and
determining a probability of disease distribution for one or more diseases for the prospective embryo based on the polygenic risk score set.

2. The method of claim 1, further comprising converting each polygenic risk score to a relative risk of disease based on the polygenic risk score.

3. The method of claim 2, wherein converting each polygenic risk score to the relative risk of disease further comprises:
calculating, using an effect size model, an odds ratio for the polygenic risk score; and
determining the relative risk of disease based on the odds ratio and a prevalence of disease associated with a particular disease.

4. The method of any of claims 1 to 3, further comprising determining one or more risk thresholds for each disease.

5. The method of claim 4, further comprising determining a percentage of the probability of disease distribution for a disease which satisfies the one or more risk thresholds corresponding to the disease.

6. The method of any of claims 1 to 5, further comprising normalizing, based on population data, each polygenic risk score in the polygenic risk score set to produce a normalized polygenic risk score set, wherein determining the probability of disease distribution is based on the normalized polygenic risk score set.

7. The method of claim 6, wherein population data comprises ancestry specific population data.

8. The method of any one of claims 1 to 7, further comprising:
generating, using a meiotic recombination model, a maternal gamete based on the phased maternal chromosome set and the one or more meiotic recombination sites of interest;
generating, using the meiotic recombination model, a paternal gamete based on the phased paternal chromosome set and the one or more meiotic recombination sites of interest; and
generating the one or more simulated embryo genotypes based on the paternal gamete and the maternal gamete.

9. The method of any of claims 1 to 8, further comprising:
obtaining a maternal genome from a maternal subject and a paternal genome from a paternal subject;
phasing the maternal genome to generate the phased maternal chromosome set; and
phasing the paternal genome to generate the phased paternal chromosome set.

10. The method of claim 9, wherein phasing of the maternal genome or paternal genome is performed using one or more of population-based methods or molecular based methods.

11. The method of either of claim 9 or 10, further comprising:
performing whole genome sequencing on a biological sample obtained from the maternal subject to determine the maternal genome; and
performing whole genome sequencing on a biological sample obtained from the paternal subject to determine the paternal genome.

12. The method of any of claims 9 to 11, further comprising:
determining sibling genomic information;
generating the phased maternal chromosome set based on the maternal genome and the sibling genomic information; and
generating the phased paternal chromosome set based on the paternal genome and the sibling genomic information.

13. The method of any of claims 9 to 12, further comprising:
obtaining population genotype data comprising individual genotypes for a plurality of unrelated individuals;
generating the phased maternal chromosome set based on the maternal genome and the population genotype data; and
generating the phased paternal chromosome set based on the paternal genome and the population genotype data.

14. The method of any of claims 9 to 13, further comprising:
determining sibling genomic information; and
determining the one or more meiotic recombination sites of interest based on the sibling genome, the maternal genome, and the paternal genome.

15. The method of any of claim 12 or 14, wherein:
sibling genomic information is determined using at least one of array measurements, next-generation sequencing, or whole genome sequencing, and
sibling genomic is obtained from at least one of a sibling embryo, a full biological sibling, or a half biological sibling.

16. The method of any of claims 1 to 15, wherein chromosome-length parental haplotypes are obtained across an entire genome for each simulated embryo.

17. The method of any of claims 1 to 16, further comprising:
generating an additional in-vitro fertilization (IVF) cycle recommendation based on the probability of disease distribution for one or more diseases for the prospective embryo; and
outputting the IVF cycle recommendation.

18. The method of claim 17, further comprising:
determining a disease occurrence risk based on the probability of disease distribution, wherein the IVF cycle recommendation is based on the disease occurrence risk.

19. The method of claim 18, wherein the additional IVF cycle recommendation is indicative of whether to perform an additional round of IVF.

20. An apparatus for determining a probability of disease distribution associated with a prospective embryo, the apparatus comprising a processor and a memory storing software instructions that, when executed by the processor, cause the apparatus to perform the steps recited in any of claims 1 to 19.

21. A computer program product for determining a probability of disease distribution associated with a prospective embryo, the computer program product comprising at least one non-transitory computer-readable storage medium storing software instructions that, when executed by an apparatus, cause the apparatus to perform the steps recited in any of claims 1 to 19.
